(19) United States
(12) Patent Application Publication
(10) Pub. No.: US 2003/0138412 A1
(43) Pub. Date: Jul. 24, 2003

(54) Inhibition of tumor growth and metastasis by N5 gene

(76) Inventors: David W. Goodrich, East Aurora, NY (US); Shenmin Yin, New York, NY (US); Jaleh Doostzadeh, Fremont, CA (US)

Correspondence Address: Priya D. Subramony, Fullbright & Jaworski L.L.P., Suite 2400, 600 Congress Avenue, Austin, TX, 78701, US

(21) Appl. No.: 10/186,185

(22) Filed: Jun. 27, 2002

Related U.S. Application Data
(60) Provisional application No. 60/301,619, filed on Jun. 28, 2001.

(51) Int. Cl. A61K 48/00; G01N 33/574

(52) U.S. Cl. 424/93.21; 435/7.23; 514/44

(57) ABSTRACT

The present invention concerns methods for treating pancreatic and ovarian cancers in a subject. These methods employ compositions comprising the N5 gene product, p84N5 and include nucleic acids and proteins/peptides or polypeptides encoding p84N5 or portions thereof. The invention also concerns prognostic applications wherein the levels of expression of p84N5 have been correlated to sensitivity to radiation treatments and/or chemotherapeutic agents. Therefore, the invention also concerns methods for prescribing a specific therapeutic regimen comprising specific radiation and chemotherapy doses and adjustments in such doses based on the individual patients p84N5 expression levels.

The present application claims priority to co-pending U.S. Application Serial No. 60/301,619 filed Jun. 28, 2001. The entire contents of the above-referenced application are incorporated herein by reference. The government owns rights in the present invention pursuant to grant numbers CA-70292-01 and CA-16672 from the National Institutes of Health.

### BACKGROUND OF THE INVENTION

1. Field of the Invention

The present invention relates generally to the fields of cancer and biochemistry. More particularly, it concerns methods for treating and preventing cancer comprising induction of programmed cell death or apoptosis in tumor cells by the p84N5 gene in pancreatic and ovarian cancers. The invention also concerns prognostic methods that allow optimization of radiation and/or chemotherapeutic-based treatment regimens for a patient based on determining the levels of expression of p84N5 in the cancer cells of that patient.

2. Description of Related Art

Pancreatic adenocarcinoma is the fifth leading cause of cancer death in America, with about 27,000 new cases and 25,000 cancer related deaths per year (Evans et al., 1997). Only 3% of all patients diagnosed with pancreatic cancer can expect to survive 5 years. At the time of diagnosis, patients usually have locally advanced or metastatic disease to the lymph nodes, liver, lungs and peritoneum (Evans et al., 1997; Korc, 1998). The same is true for certain forms of ovarian cancer which are also diffucult to treat. The use of traditional chemotherapy and radiation has generated only modest improvements in outcome after resection and likewise has offered little hope to those individuals with unresectable disease (Jacobson et al., 1997). Given this background, new forms of cancer therapy are required to improve treatment outcomes.

Cancer is a result of defects in the coordination of cell proliferation and programmed cell death. The extent of cell death is physiologically controlled by activation of a programmed suicide pathway that results in a morphologically recognizable form of death termed apoptosis (Jacobson et al., 1997; Vaux et al., 1994; Favrot et al., 1998). Since the benefit of traditional therapy often relies on triggering the apoptotic death of tumor cells, gene therapy-mediated induction of apoptosis or restoration of apoptotic sensitivity in tumors may provide potentially effective means to treat cancer in general (Favrot et al., 1998) and pancreatic adenocarcinoma in particular (Clary and Lyerly, 1998; Aspinall and Lemoine, 1999). Although treating cancer by p53 gene therapy has received intense interest because pre-clinical and phase I clinical trials have yielded promising therapeutic effects in a number of cancers (Clayman, 2000; Kigawa and Terakawa, 2000; Chen and Mixson, 1998; Roth et al., 1999; Swisher et al., 1999), some tumor cells are insensitive to p53 gene therapy, particularly those that express wild-type p53 or overexpress mdm2 (Harris et al., 1996; Meng et al., 1998; Vinyals et al., 1999). Hence, the effectiveness of p53 gene therapy may be dependent on the status of the p53 regulatory pathway within individual tumors. For example, while adenoviral-mediated p53 gene therapy has been demonstrated to suppress the growth of human pancreatic cancer cells by induction of apoptosis (Bouvet et al., 1998), transduction of the p53 gene does not inhibit the growth or increase chemosensitivity of all pancreatic cancer cell lines, suggesting that p53 gene therapy alone may not consistently produce beneficial therapeutic effects (Kimura et al., 1997). To maximize the effectiveness of gene-based therapies, identifying candidate genes that play a role in a variety of different cancers and in distinct apoptotic pathways would be beneficial.

The present inventors have previously demonstrated that adenoviral-mediated gene transfer of nucleic acids encoding a gene called N5, reduced the growth of osteosarcoma, breast carcinoma, and ovarian carcinoma cells in vitro by induction of apoptosis (co-pending U.S. Patent Application Serial No. 60/151,687, incorporated herein by reference; and Yin et al., 2000). The N5 gene was previously cloned based on the ability of its encoded protein to bind an amino-terminal domain of the retinoblastoma tumor suppressor gene (Rb) product (Durfee et al., 1994). The N5 protein (p84N5) contains sequence similarity to the death domains of other proteins involved in the regulation of apoptosis (Feinstein et al., 1995).

Apoptosis induced by expression of p84N5 is dependent on an intact death domain and is inhibited by coexpression of Rb (Doostzadeh-Cizeron et al., 1999). Further, p84N5-mediated apoptosis is associated with caspase-6 activation, NF-κB activation, activation of a G2/M cell cycle checkpoint, and does not require p53 (co-pending U.S. Patent Application Serial No. 60/151,687; Doostzadeh-Cizeron et al., 2000a; Doostzadeh-Cizeron et al., 2000b, all of which are incorporated herein by reference). In addition, p84N5 is localized in the nucleus. As Rb inhibits p84N5 induced apoptosis, tumors deregulated for Rb growth control may be particularly sensitive to the effects of N5 gene therapy.

Adenoviral-mediated N5 (AdN5) gene transfer, activates an apoptotic pathway that is distinct from that activated by adenoviral-mediated p53 (Adp53) gene transfer (Le et al., 1998; Liu et al., 1994; Hedlund et al., 1999). For example, Adp53 infection induces a G1 cell cycle arrest and subsequent apoptosis (Meng et al., 1998) while AdN5 causes a G2/M cell cycle arrest followed by apoptosis (co-pending U.S. Patent Application Serial No. 60/151,687; Doostzadeh-Cizeron et al., 2000a). AdN5 treatment also activates NF-κB (co-pending U.S. Patent Application Serial No. 60/151,687; Yin et al., 2000; Doostzadeh-Cizeron et al., 2000a) while Adp53 inhibits this activation. Although N5 contains sequence similarity to other death domain containing proteins that induce apoptosis, it is likely to function differently since it is localized exclusively to the nucleus during interphase. Thus, N5 induces a p53-independent nuclear apoptotic pathway.

As current cancer therapies have only limited therapeutic benefits, especially with regard to pancreatic and ovarian cancers and metastatic cancers, there exists a need for a treatment that is specific for such tumor cells that do not have side effects of chemotherapeutic agents.

### SUMMARY OF THE INVENTION

The present invention overcomes the existing defects in the art and provides an N5 gene based therapy that effectively suppresses tumor growth and metastasis in pancreatic adenocarcinoma and ovarian cancers.

Co-pending U.S. Patent Applications, Serial Nos. 60/151,687 and 09/653,465, the entire disclosures of which are incorporated herein by reference, describes nucleic acid and protein compositions comprising the N5 gene product, p84N5 and its activities and functions. p84N5 contains a functional death domain, can interact with the retinoblastoma gene product, and is normally localized in the nucleus of cells. Increasing the activity level of p84N5 in cancer cells is therefore beneficial for the treatment of cancer.

The inventors have demonstrated by in vivo studies in subcutaneous and orthotopic mouse models of human panceratic and human ovarian cancers that N5 gene based therapy effectively suppresses tumor growth as well as metastasis.

Thus, the present invention provides methods of inducing apoptosis in a pancreatic or an ovarian cancer cell comprising contacting the cell with a recombinant vector encoding a p84N5 death domain operably linked to a promoter that functions in the cell.

In some embodiments of the method, the pancreatic cancer cell can be a pre-cancerous pancreatic cell, a metastatic pancreatic cell, or a malignant pancreatic cell. Pancreatic cancer is histologically subdivided into the following malignant sub-types: ductal adenocarcinoma; mucinous cystadenocarcinoma; acinar carcinoma; unclassified large cell carcinoma; small cell carcinoma; pancreatoblastoma. In addition, uncertain malignant subtypes of pancreatic cancer include intraductal papillary neoplasm; mucinous cystadnoma; and papillary cystic neoplasm. Although not limited to these examples, the methods of the present invention can be used to induce apoptosis in these as well other types of pancreatic cancer cells.

In other embodiments, the methods of inducing apoptosis are also useful in the context of ovarian cancer cells. The ovarian cancer cell can be a pre-cancerous ovarian cell, a metastatic ovarian cell, or a malignant ovarian cell. In non-limiting examples, ovarian cancer is histologically subdivided into serous, mucinous, endometrioid, clear cell mesonephroid, Breiner, or mixed epithelial cancer and the methods of the present invention can be used to induce apoptosis in these as well other types of ovarian cancer cells.

The N5 gene can induce the apoptosis in tumor cells that lack functional caspase-3, such as MCF-7, (co-pending U.S. Patent Application Serial No. 60/151,687; co-pending U.S. patent application Ser. No. 09/653,465; Yin et al., 2000). Therefore, in some embodiments of the method, the cancer cell lacks functional caspase-3.

In other embodiments of the method, the cancer cell is retinoblastoma-negative. In still other embodiments, the cancer cell is comprised in a tumor. In specific embodiments of the method, the cell is comprised in an animal. In other specific aspects, the animal is human.

Therefore, in some embodiments, the method is further defined as a method of treating cancer. In other embodiments the method is further defined as a method of preventing cancer.

In one aspect of the method, the p84N5 death domain comprises a p84N5 sequence up to and including full length p84N5 sequences. In other aspects, the p84N5 death domain comprises the sequence shown in SEQ ID NO: 1.

In one embodiment, the contacting comprises administering the recombinant vector encoding the p84N5 death domain. In specific embodiments, the recombinant vector can be a viral vector. In specific embodiments, the viral vector is an adenoviral vector, an adeno-associated viral vector, a retroviral vector, a lentiviral vector, a herpes viral vector, a papilloma viral vector, or a hepatitis B viral vector. One of skill in the art will recognize that the invention is not limited to the type of viral vector and all kinds of ciral vectors known in the art may be used. In such embodiments, the viral vector may further be either replication-defective or replication-competent.

In embodiments where the viral vector is an adenovirus, the adenovirus maybe administered at a dose of about 10¹⁰ to about 10¹³ pfu. As will be recognized by the skilled artisan, the final dosage administered to a patient will be subject to further adjustments based on specific disease conditions, age, gender, and other health conditions of each individual patient, and such dose adjustments will be performed by a trained physician at the time of treatment. The present invention is therefore not limited by the dose related adjustments.

The invention also contemplates combination therapies in conjunction with the p84N5 based gene therapy. Levels of endogenous p84N5, in a subset of tumor cell lines (n=6), were found to correlate to sensitivity to ionizing radiation, indicating that N5 gene therapy restores and/or enhances sensitivity of tumor cells to genotoxic agents. Therefore, in some embodiments, the method further comprises treating the subject with a second agent, wherein the second agent is a therapeutic polypeptide, another nucleic acid encoding a therapeutic polypeptide, a chemotherapeutic agent, an immunotherapeutic agent, or a radiotherapeutic agent. For example, in some embodiments, the therapeutic polypeptide and/or therapeutic nucleic acid may encode p53. Other adjunct cancer therapies such as surgery, tumor resection, heat therapies, hormonal therapy, etc., are also contemplated. In such embodiments, the second agent may be administered simultaneously with the recombinant vector encoding p84N5. Alternatively, the second agent may be administered at a different time than the recombinant vector encoding p84N5. Thus, the second agent may be administered prior to the or after the p84N5-based therapy.

Various modes of administering the recombinant vector encoding p84N5 are contemplated. Some exemplary modes of administration include, intravenous, intraartetial, intraperitoneal, intradermal, intratumoral, intrathecal, intramuscular, oral, dermal, nasal, buccal, rectal, vaginal, inhalation, or topical administration.

The invention also provides methods of inhibiting cell division of a pancreatic or an ovarian cancer cell, comprising contacting the cell with a recombinant vector encoding a p84N5 death domain operably linked to a promoter that functions in the cell.

In addition, the invention also provides methods of inhibiting the growth of a pancreatic or an ovarian cancer cell comprising contacting the cell with a recombinant vector encoding a p84N5 death domain operably linked to a promoter that functions in the cell. In one embodiment of this method the growth is metastatic growth.

The invention also provides methods of inhibiting the metastatic potential of a pancreatic or an ovarian cancer cell comprising contacting the cell with a recombinant vector encoding a p84N5 death domain operably linked to a promoter that functions in the cell.

Further provided are methods for reducing tumor burden of a pancreatic or an ovarian cancer cell comprising contacting the cell with a recombinant vector encoding a p84N5 death domain operably linked to a promoter that functions in the cell.

Additionally, the invention provides methods of inducing tumor regression of a pancreatic or an ovarian cancer cell, comprising contacting the cell with a recombinant vector encoding a p84N5 death domain operably linked to a promoter that functions in the cell.

In other embodiments, the invention provides methods of killing a pancreatic or an ovarian cancer cell, comprising contacting the cell with a recombinant vector encoding a p84N5 death domain operably linked to a promoter that functions in the cell.

In yet other methods, the invention provides methods of increasing sensitivity to chemotherapy or radiotherapy of a pancreatic or an ovarian cancer cell, comprising contacting the cell with a recombinant vector encoding a p84N5 death domain operably linked to a promoter that functions in the cell.

In still other embodiments, the invention provides methods of inducing apoptosis in a pancreatic or an ovarian cancer cell comprising contacting a cell with a p84N5 protein, peptide, or polypeptide. The protein, peptide, or polypeptide may be produced by automated synthetic methods, by recombinant DNA methods, or may be a purified, partially purified or substantially purified composition.

Treating cancer is defined as inducing apoptosis, inhibiting cell division, inhibiting metastatic potential, reducing tumor burden, increasing sensitivity to chemotherapy or radiotherapy, killing a cancer cell, inhibiting the growth of a cancer cell, or inducing tumor regression in a subject. In preferred embodiments, the subject is human.

The invention also provides prognostic methods for determining an optimal treatment regimen for cancers in general. The present inventors have demonstrated a correlation between the expression levels of p84N5 proteins or polypeptides or peptides or even mRNA in cancer cells and sensitivity of the cancer cells to radiotherapeutic and/or chemotherapeutic agents. An increased expression of p84N5 correlates to an increased sensitivity of the cancer to radio- and/or chemo- therapeutic agents. These methods allow the prediction of success of a chemo- and/or radio- therapeutic regimen in a patient.

Therefore, in one embodiment, the invention further provides methods for a cancer therapy comprising a) obtaining a biological sample from a cancer patient; b) detecting the level of expression of a p84N5 polypeptide or protein or peptide in the biological sample; c) determining the dosage of a radiotherapeutic agent and/or a chemotherapeutic agent to be administered to the patient based on the level of expression of the p84N5 polypeptide or protein or peptide; and d) providing to the patient the determined dosage of radiotherapeutic agent and/or chemotherapeutic agent.

In another embodiment, the invention also provides methods for a cancer therapy comprising a) obtaining a biological sample from a cancer patient; b) detecting the level of expression of a p84N5 encoding nucleic acid in the biological sample; c) determining the dosage of a radiotherapeutic agent and/or a chemotherapeutic agent to be administered to the patient based on the level of expression of the p84N5 nucleic acid; and d) providing to the patient the determined dosage of radiotherapeutic agent and/or chemotherapeutic agent.

In some embodiments, such methods further comprise comparing the level of expression of the p84N5 polypeptide or protein or peptide or nucleic acid in the biological sample with the level of expression of the p84N5 polypeptide or protein or peptide in a control biological sample. The control biological sample may be from a cancer with a known sensitivity to the radiotherapeutic agent or the chemotherapeutic agent.

In other embodiments, the prognostic evaluation may require comparison of the level of the p84N5 protein, polypeptide or peptide or mRNA in the biological cancer sample to be compared to a control biological sample which may be comprised of normal cells, and/or comprised of cells of other patients afflicted with a similar cancer, and/or comprised of cells obtained at an earlier stage of treatment of the same patient.

It is contemplated that these prognostic methods will be applicable to cancer patients afflicted with different types of cancers, such as but not limited to, hematological cancers thyroid cancers, melanomas, T-cell cancers, B-cell cancers, breast cancers, an ovarian cancers, pancreatic cancers, prostate cancers, colon cancers, bladder cancers, lung cancers, liver cancers, stomach cancers, testicular cancers, an uterine cancers, brain cancers, lymphatic cancers, skin cancers, bone cancers, kidney cancers, rectal cancers, or sarcomas.

The present invention also provides kits that comprise reagents to detect the expression levels of p84N5 products in a biological sample. Thus, in one embodiment, the invention provides a kit for detecting the levels of a p84N5 polypeptide/peptide/protein in a biological sample comprising; a) at least one antibody with immunospecificity to a p84N5 polypeptide/peptide/protein; b) an immunodetection reagent; and c) reagents and buffers, enclosed in a suitable container means. In another embodiment, the invention provides a kit for detecting the levels of a p84N5 mRNA in a biological sample comprising; a) at least two primers that hybridize to a sequence comprising SEQ ID NO: 1 or a fragment or a region thereof; and b) reagents, buffers and enzymes, enclosed in a suitable container means.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. N5 gene transfer inhibits cell proliferation in vitro by induction of apoptosis. An equal number of human pancreatic adenocarcinoma cells were plated and treated at an MOI of 50 with the indicated virus or PBS. Aliquots of the infected cells were harvested at the indicated times and the number of cells excluding trypan blue counted using a hemacytometer. The data presented are the mean of three infections. The standard deviations from the mean are smaller than the symbols representing the data points.

FIGS. 2A & 2B. Tumor cells are more sensitive to the effects of AdN5 than normal cells. (FIG. 2A.) An equal number of the indicated tumor cells were plated and infected as in FIG. 1. At the indicated times post-infection, cells were harvested and the percentage of viable cells determined by trypan blue dye exclusion. The data are the mean of three infections; the standard deviations from the mean are smaller than the symbols representing the data points. (FIG. 2B.) An equal number of WI38 cells were plated and treated as indicated. At the indicated times post-infection, the number of viable cells (trypan blue dye excluding) were counted using a hemacytometer. The data are the mean of three infections; the standard deviations from the mean are smaller than the symbols representing the data points.

FIGS. 3A & 3B. Intratumoral injection of AdN5 inhibits tumor cell proliferation in vivo. (FIG. 3A) Equal numbers SKOV3-ip1 cells were subcutaneously implanted in nude mice. One to two weeks later, palpable tumors were treated by intratumoral injection with the indicated virus. Tumor volumes were measured weekly after implantation. Each data point represents the mean and standard deviation of the tumor volumes for at least six mice for each virus. (FIG. 3B) FG human pancreatic adenocarcinoma cells were implanted and treated as above. The data presented represent the mean and standard deviation of at least six mice for each treatment.

FIGS. 4A & 4B. Increased radiation sensitivity due to the expression of N5 gene products. (FIG. 4A) Radiation sensitivity correlates with levels of N5 protein in different cancer cell lines. An equal mass of total cell protein extracted from each of the indicated cell lines was analyzed for p84N5 by western blotting. (FIG. 4B) Radiation clonogenic survival curves for the indicated cell lines are shown. The data points are the mean of assays done in triplicate.

FIG. 5. Efficiency of adenovirus-mediated gene transduction. SKOV3-ip1 and FG human pancreatic adenocarcinoma cells were treated with AdGFP at MOIs ranging from 1 to 100. The percentages of GFP positive cells were scored for each sample by fluorescence microscopy. The results presented are the mean of the three infections. The standard deviation from the mean is smaller than the symbols representing the data.

FIG. 6. Mapping of amino acids required for exclusive nuclear localization of p84N5. An 1800 bp fragment encoding amino acids 54 to 656 of the p84N5cDNA was subcloned in frame into the EGFP-C1 mammalian ex-pression vector to create GFPN5. Subsequent deletion mutants were derived from this construct as follows: GFPN5NB was created by deleting the NheI-BamHI fragment of GFPN5; GFPN5EB was created by deleting the EcoRI-BamHI fragment of GFPN5; GFPN5SB was created by deleting the SalI-BamHI fragment of GFPN5; GFPN5SE is an inframe internal deletion created from GFPN5EB by removing the SalI-EcoRI fragment and inserting a SpeI linker. GFP is expressed from the empty EGFP-C1 plasmid. The schematic indicates the p84N5 amino acids included within each plasmid, the position of the p84N5 death domain, and whether the expressed fusion protein is localized predominantly within the nucleus.

FIG. 7. Identification of the p84N5 nuclear localization signal. The mutant plasmids were constructed by PCR-mutagenesis as previously described (Fisher and Pei, 1997). The template for mutagenesis was GFPN5.GFPN5-NLS was created using the following pair of adjacent phosphorylated primers (5′-AATTATTCTCGTAGGTTTGGTA-TCTGATG-3′ (SEQ ID NO. 6) and 5′-ATTCTGACGGGAAATGAGGAGTTA-ACAAGG-3′ (SEQ ID NO. 7)). GFPN5+NLS was created using the following pair of adjacent phosphorylated primers (5′-CATCTCCTGGGCAT-AACGAATTATTCTCGTAGGTTTGGTATC-3′ (SEQ ID NO. 8) and 5′-GAA-GGCGAAGAAGAAGCCATTCTGACGGGAAATGAGGAG-TTA-3′ (SEQ ID NO. 9)). GFPN5+NES was created using the following pair of adjacent phosphorylated primers (5′-GAACTTCTTCGTCATAATTATTCTCGTAGGTTTGGTATC-3′ (SEQ ID NO. 10) and 5′-GGCACGCTCACGATCATTCTGACGGGAAATGAGGAG-3′ (SEQ ID NO. 11)). DNA sequencing was used to confirm the mutagenesis. The schematic indicates the amino acid sequence of the putative bipartite p84N5 NLS, the amino acids contained in the mutant derivatives, and whether they are localized predominantly within the nucleus.

FIG. 8. Nuclear localization is required for N5-induced apoptosis. C33-A cells were analysed for apoptosis by annexin V staining upon expression of the indicated fusion proteins. Cells were seeded in 10 cm dishes at 25% confluency and transfected the following day as in FIG. 1. Thirty-six hours after transfection cells were harvested by trypsinization, washed with PBS, and stained with Annexin V-R-PE as per the manufacturer's instructions (Caltag Laboratories, Burlingame, Calif., USA). FACS analysis was performed on a FACSCalibur instrument (Becton Dickinson, San Jose, Calif., USA). Successfully transfected cells were gated based on GFP fluorescence and the percentage of these cells po-sitive for annexin V determined using the PE channel. In parallel experiments under the same experimental conditions, the percentage of late apoptotic or necrotic cells as determined by propidium iodide staining was typically less than 5%. The data shown are the mean and standard deviation of at least three transfections Death domain protein p84N5 functions within the nucleusRL Evans et al3oncogene FIG. 9. Nuclear localization is required for p84N5-induced loss of clonogenic potential. SAOS-2 cells were seeded in 100-mm dishes at 25% confluency and transfected the following day using the calcium phosphate precipitation method as in FIG. 6 using 12 mg of total DNA, including 2 mg of EGFP-C1 plasmid (Clontech, Palo Alto, Calif., USA). The day after transfection, cells wereexamined for green fluorescent protein (GFP) by fluorescence microscopy and the number of GFP positive cells per randomly chosen fields of view were counted. Transfection efficiencies for independent assays ranged between 10±15%, but transfection efficiencies among different plasmids within an experiment were notconsistently different. Two days following transfection, cells weregrown in the presence of 500 mg/ml G418 to select for successfully transfected cells. Two weeks later, the number of surviving GFP-positive colonies containing greater than 20 cells were counted in at least 50 randomly chosen fields of view using a fluorescence microscope. The surviving fraction was calculated by dividing the number of colonies per field of view by the number of GFP positive cells per field of view originally recorded one day after transfection. The data shown are the mean and standard deviation of at least three independent experiments for each plasmid.

### DESCRIPTION OF ILLUSTRATIVE EMBODIMENTS

Pancreatic cancers and ovarian cancers are associated with poor patient prognosis and a high incidence of mortality. Therefore, pancreatic and ovarian cancers pose a challenge as they are in general resistant to currently existing treatment modalities.

The current inventors demonstrate herein that adenovirus mediated N5 gene (AdN5) transfer inhibits the growth of ovarian and pancreatic tumors in vitro and in vivo. Significant reduction in tumor cell growth rate was observed upon AdN5 infection relative to a control comprising a adenoviral construct bearing the green fluorescent protein (GFP) gene (AdGFP) treated cells. These results were observed both in vitro in cancer cell lines and in in vivo studies using mouse models where tumor volume and tumor mass reductions were observed of human cancers. In addition, AdN5 treatment reduced the incidence and mean number of liver metastasis observed in the orthotopic mouse model of pancreatic cancer.

The present invention therefore provides methods for the treatment of pancreatic and ovarian cancers using p84N5. p84N5 encoding nucleic acids or p84N5 proteins can be administered by different modes to a cancer patient, such that these patients are conferred a therapeutic benefit as a result of the treatment. The term “therapeutic benefit” used herein refers to anything that promotes or enhances the well-being of the patient with respect to the medical treatment of the patient's cancer. A list of nonexhaustive examples of this includes extension of the patient's life by any period of time; decrease or delay in the neoplastic development of the disease; decrease in hyperproliferation; reduction in tumor growth; delay or prevention of metastases; reduction in the proliferation rate of a cancer cell or tumor cell; induction of apoptosis in any treated cell or in any cell affected by a treated cell; induction of cell killing; a decrease in cell growth; and/or a decrease in pain to the patient that can be attributed to the patient's condition.

The result of this treatment can be the induction of apoptosis, inhibition of cell division, inhibition of metastatic potential, reduction of tumor burden, increased sensitivity to chemotherapy or radiotherapy, killing of a cancer cell, inhibition of the growth of a cancer cell, induction of tumor necrosis, and induction of tumor regression of a pancreatic or an ovarian cancer cell.

The present inventors have also demonstrated that fast growing (FG) pancreatic tumor cells are more sensitive to AdN5 than they are to Adp53. Therefore, N5 gene therapy may be effective in tumors that are relatively insensitive to p53 gene therapy.

The inventors have also shown that the expression levels of p84N5 polypeptide(s)/protein(s)/peptide(s) and/or mRNA are correlated to the radiation sensitivity of cancer cells. Thus, cancer cells or cell lines that express higher levels of p84N5 products are more susceptible to radiation treatments. The inventors envision that the same will be true for chemotherapeutic sensitivity of cancer cells as well. It is therefore contemplated, that determining the levels of expression of p84N5 will provide a prognostic test that will allow a physician to determine an optimal dose of radiation and/or chemotherapy that a particular cancer will respond to.

This prognostic test is contemplated useful both in combination with the gene therapy treatments provided herein for pancreatic and ovarian cancers, which results in increased p84N5 expression in cancer cells, as well as for prescribing doses of radiation and/or chemotherapy for patients suffering from other types of cancers. One can obtain a sample of the cancer tissue, such as by means of a biopsy sample, a blood sample, etc. and determine the expression of p84N5 proteins and/or mRNA levels in that cancer. The level of expression will determine whether the patient should receive a higher or lower dose of radiation and/or chemotherapeutic agent. Additionally, kits are contemplated that provide the necessary reagents and components to detect levels of p84N5 in a clinical cancer sample.

I. Retinoblastoma Gene Product

p84N5 is known to interact with the retinoblastoma gene product (Rb), to be normally localized to the nucleus of cells and is shown in the present invention to contain a functional death domain. These properties are important attributes of the present invention and are discussed in greater detail in the following sections and examples.

The retinoblastoma gene (Rb), the first tumor suppressor gene identified, encodes a nuclear phosphoprotein which is ubiquitously expressed in vertebrates (Friend et al., 1986; Lee et al., 1987b; Fung et al., 1987). Mutations of this gene which lead to inactivation of its normal function have been found not only in 100% of retinoblastomas, but also in many other adult cancers including small cell lung-carcinoma (Harbour et al., 1988; Yokota et al, 1988), osteosarcoma (Toguchida et al., 1988), bladder carcinoma (Horowitz et al., 1989), prostate carcinoma (Bookstein et al., 1990a) and breast cancer (Lee et al., 1988). Reconstitution of a variety of Rb-deficient tumor cells with wild-type Rb leads to suppression of their neoplastic phenotypes including their ability to form tumors in nude mice (Huang et al., 1988; Sumegi et al., 1990; Bookstein et al., 1990b; Lee et al., 1999; Goodrich et al., 1992; Takahashi et al., 1991; Chen et al., 1992). These results provide direct evidence that Rb protein is an authentic tumor suppressor.

Rb performs its function at the early G1/G0 phase of the cell cycle as substantiated by several observations: first, the phosphorylation of Rb, presumably by members of the Cdk kinase family (Lin et al., 1991; Lee et al., 1991), fluctuates with the cell cycle (Chen et al., 1989; Buchkovich et al., 1989; DeCaprio et al., 1989); second, the unphosphorylated form of Rb is present predominantly in the G0/G1 stage (Chen et al., 1989; DeCaprio et al., 1989); third, microinjection of the unphosphorylated Rb into cells at early G1 phase inhibits their progression into S phase (Goodrich et al., 1991). These observations suggest that Rb may serve as a critical regulator of entry into cell cycle and its inactivation in normal cells could lead to deregulated growth.

Two known biochemical properties of the Rb protein have been described; one is its intrinsic DNA binding activity which was mapped to its C-terminal 300 amino acid residues (Lee et al., 1987b; Wang et al., 1990); another is its ability to interact with several oncoproteins of the DNA tumor viruses (DeCaprio et al., 1988; Whyte, et al., 1988; Dyson et al., 1989). This interaction was mapped to two discontinuous regions at amino acids 379-545 and 575-678, designated as the T-binding domains (Hu et al., 1990; Huang et al., 1990). Interestingly, mutations of the Rb proteins in tumors were frequently located in these same regions (Bookstein and Lee, 1991). These results imply that the T-binding domains of Rb proteins are functionally important and the interaction of Rb with these oncoproteins may have profound biological significance. The identification of cellular proteins that mimic the binding of T to Rb revealed a potentially complicated network. Several proteins including c-myc (Rustgi et al., 1991), Rb-p1, p2 (Defeo-Jones et al., 1991) and other proteins (Kaelin et al., 1991; Lee et al., 1991; Huang et al., 1991) have been shown to bind to Rb in vitro. At the present time, approximately 60 proteins have been implicated in binding Rb protein.

Retinoblastoma is a neoplastic condition of the retinal cells, observed almost exclusively in children between the ages of 0 and 4 years. It affects between 1 in 34,000 and 1 in 15,000 live births in the United States (Zimmerman, 1985). If untreated, the malignant neoplastic retinal cells in the intraocular tumor travel to other parts-of the body, forming foci of uncontrolled growth which are always fatal. The current treatment for a retinoblastoma is enucleation of the affected eye if the intraocular tumor is large; for small intraocular tumors, radiation therapy, laser therapy, or cryotherapy is preferred. There is no known successful treatment for metastatic retinoblastoma. As with most cancers, morbidity and mortality are reduced if diagnosis can be made early in the course of the disease.

In 30-40% of cases of retinoblastoma, the affected individual carries a heritable predisposition to retinoblastoma and can transmit this predisposition to his or her offspring as a dominant trait (Knudson, 1971). Carriers of this retinoblastoma-predisposing trait are at a greatly elevated risk for development of several other forms of primary cancer, notably osteosarcoma and soft-tissue sarcoma.

II. Death Domains and Apoptosis

The present invention concerns methods for treating pancreatic and ovarian cancers using nucleic acids encoding a p84N5 gene product and/or a p84N5 protein. In preferred embodiments, the p84N5 gene product sequence or protein sequence contains a functioning death domain. The “death domain” is defined as that portion of p84N5 that, when expressed or introduced into a tumor cell, induces apoptosis, inhibits cell division, inhibits metastatic potential, reduces tumor burden, increases sensitivity to chemotherapy or radiotherapy, kills a cancer cell, inhibits the growth of a cancer cell, or induces tumor regression.

A “death domain” has been identified in the C-terminal region of the p84N5 protein, from about amino acid 568 to about amino acid 657 of SEQ ID NO:2, based on sequence alignment (Feinstein et al., 1995). However, the a functional death domain may be larger or smaller than that defined by this alignment. A functional death domain in p84N5 may therefore be only a small portion of the protein, from about 10 amino acids to about 15 amino acids, or from about 20 amino acids to about 25 amino acids, or from about 30 amino acids to about 35 amino acids, or from about 40 amino acids to about 45 amino acids, or from about 50 amino acids to about 55 amino acids, or from about 60 amino acids to about 70 amino acids, or from about 80 amino acids to about 90 amino acids, or about 100 amino acids in length.

Alternatively, a functional death domain, as defined above, may require a larger portion of the p84N5 protein than that simply defined by sequence alignment. A portion of p84N5 from about 110 amino acids to about 115 amino acids, or from about 120 amino acids to 130 amino acids, or from about 140 amino acids to about 150 amino acids, or from about 160 amino acids to about 170 amino acids, or from about 180 amino acids to about 190 amino acids, or from about 200 amino acids to about 250 amino acids, or from about 300 amino acids to about 350 amino acids, or from about 400 amino acids to about 450 amino acids, or from about 500 amino acids to about 600 amino acids, or the full length protein as defined in SEQ ID NO:2 may be required for function.

The p84N5 death domain, while identified based on sequence alignment (Feinstein et al., 1995), is further defined herein functionally. A p84N5 death domain is the minimum region of p84N5 that is necessary and sufficient for the generation of cytotoxic death signals, anti-viral responses (Tartaglia et al., 1993), and/or the activation of acid sphingomyelinase (Wiegmann et al., 1994) when overexpressed or ectopically expressed in cells. A functional p84N5 death domain, when overexpressed or ectopically expressed in a cancer cell, is further defined as being capable of inducing apoptosis, inhibiting cell division, inhibiting metastatic potential, reducing tumor burden, increasing sensitivity to chemotherapy or radiotherapy, killing a cancer cell, inhibiting the growth of a cancer cell, or inducing tumor regression.

The term “death domain” was originally coined in 1993 by Tartaglia et al. as a result of deletion mutagenesis studies involving TNF-R1 (p55)-mediated apoptotic cell death. These studies revealed that an 80 amino acid domain, which is localized to the C-terminal portion of the protein's intracellular region, is responsible for the generation of cytotoxic death signals, anti-viral responses (Tartaglia et al., 1993), and the activation of acid sphingomyelinase (Wiegmann et al., 1994); it also is partially responsible for, in conjunction with residues in the N-terminal portion in the intracellular region, the induction of nitric oxide (NO) synthase activity (Tartaglia et al., 1993). Homology searches have revealed that the TNF-R1 death domain is approximately 65% similar (28% identical) to a 65 amino acid region within the intracellular domain of the Fas antigen; mutagenesis studies have confirmed that these 65 amino acids are required for the induction of cell death following treatment with an anti-Fas antibody in conjunction with actinomycin D (Itoh et al., 1993). Supporting evidence for a functional overlap between the domains of these two receptors was achieved through the generation of a “death signal delivering” chimeric receptor which replaced TNF-R1 amino acid residues 324-326 with the corresponding amino acids of the Fas antigen (Tartaglia et al., 1993).

The death domain, aside from being the only homologous intracellular domain that is shared by two members of the TNFR superfamily, generates a cytotoxic signal irrespective of its position with respect to the extracellular domain (Tartaglia et al., 1993). In addition, this domain appears to mediate self-association of both TNF-R1 and Fas, thereby mimicking the aggregation of events which are induced by ligand binding to each of these receptors (Boldin et al., 1995). These results, which demonstrate that the death domain is an independent domain at both the structural and functional levels, were recently confirmed by the identification and subsequent characterization of three death domain-containing proteins, each of which can generate an apoptotic signal when overexpressed in cells.

Other proteins containing death domains have more recently been defined based on sequence alignments (Feinstein et al., 1995). These include the low affinity nerve growth factor receptor and MORTI (Boldin et al., 1995), the ankyrins (Boldin et al., 1995; Cleveland and lhle, 1995), two Drosophila proteins, PELLE (Shelton and Wasserman, 1993) and TUBE (Letsou et al., 1991), and the p84N5 protein described in the present application.

Death domains mediate protein-protein interactions with analogous death domain sequences. It is not clear whether the transduction of a cytocidal signal is the only function of death domains. Death domains probably participate in transduction of other signals through unique protein-protein interactions.

The rapid induction of cell death via the death domain is heretofore unique to TNF-R1 and Fas; however, despite the characterization of a defined “death inducing” region within each of these receptors, the intermediates involved in the transmission of their signals were, until recently, completely unknown. As with other receptors which are devoid of catalytic activity, TNF-R1 and Fas were suspected to utilize cellular protein as “downstream messengers of death.” Many charged residues that are well conserved in both proteins were suspected to be widely dispersed throughout portions of the death domains which are oriented to interact with protein components of the cytoplasm (Tartaglia et al., 1993).

To date, three death domain-containing proteins which associate with either TNF-RI or Fas have been identified and characterized with respect to their ability to induce apoptosis and other downstream signaling events which are activated in immune responses achieved through ligand binding to each of these receptors.

III. p84N5 Genes and DNA Segments

Important aspects of the present invention concern the therapeutic use of isolated DNA segments and recombinant vectors encoding p84N5, and recombinant host cells through the application of DNA technology, that express wild-type, polymorphic or allelic variants of p84N5, using the sequence of SEQ ID NO:1, and biologically functional equivalents thereof. The compositions used in the therapeutic methods described herein are disclosed in detail in co-pending U.S. Patent Application Serial No. 60/151,687, the entire disclosure of which is incorporated herein by reference.

As used herein, the term “DNA segment” refers to a DNA molecule that has been isolated free of total genomic DNA of a particular species. Therefore, a DNA segment encoding p84N5 refers to a DNA segment that contains wild-type, polymorphic or mutant p84N5 coding sequences yet is isolated away from, or purified free from, genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified p84N5 gene refers to a DNA segment including p84N5 protein coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and smaller engineered gene segments that express, or may be adapted to express, proteins, polypeptides, domains, peptides, fusion proteins and mutants.

“Isolated substantially away from other coding sequences” means that the gene of interest, in this case the p84N5 gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns the therapeutic use for pancreatic and ovarian cancers of isolated DNA segments and recombinant vectors incorporating DNA sequences that encode a p84N5 protein or peptide that includes within its amino acid sequence a contiguous amino acid sequence in accordance with, or essentially as set forth in, SEQ ID NO:2.

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:2 will be sequences that are “essentially as set forth in SEQ ID NO:2”, provided the biological activity of the protein is maintained.

In certain other embodiments, the invention concerns the therapeutic use of isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO: 1 and has relatively few codons that are not identical, or functionally equivalent, to the codons of SEQ ID NO:1. Again, DNA segments that encode proteins exhibiting p84N5 activity will be most preferred.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

Excepting intronic or flanking regions, and allowing for the degeneracy of the genetic code, sequences that have between about 70% and about 79%; or more preferably, between about 80% and about 89%; or even more preferably, between about 90% and about 99%; of nucleotides that are identical to the nucleotides of SEQ ID NO: 1 will be sequences that are “essentially as set forth in SEQ ID NO:1”.

Sequences that are essentially the same as those set forth in SEQ ID NO:1 may also be functionally defined as sequences that are capable of hybridizing to a nucleic acid segment containing the complement of SEQ ID NO: 1 under relatively stringent conditions. Suitable relatively stringent hybridization conditions will be well known to those of skill in the art, as disclosed herein.

Hybridization is understood to mean the forming of a double-stranded molecule or a molecule with partial double-stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and ionic strength of a desired stringency are determined in part by the length of the particular probe, the length and base content of the target sequences, and to the presence of formamide, tetramethylammonium chloride or other solvents in the hybridization mixture. It is also understood that these ranges are mentioned by way of example only, and that the desired stringency for a particular hybridization reaction is often determined empirically by comparison to positive and negative controls.

Accordingly, the nucleotide sequences may be used for their ability to selectively form duplex molecules with complementary stretches of genes or RNAs or to provide primers for amplification of DNA or RNA from tissues. Depending on the application envisioned, it is preferred to employ varying conditions of hybridization to achieve varying degrees of selectivity of probe towards target sequence.

For applications requiring high selectivity, it is preferred to employ relatively stringent conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and would be particularly suitable for isolating specific genes or detecting specific mRNA transcripts. It is generally appreciated that conditions may be rendered more stringent by the addition of increasing amounts of formamide.

Naturally, the present invention also encompasses the therapeutic use of DNA segments that are complementary, or essentially complementary, to the sequence set forth in SEQ ID NO:1. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein.

The nucleic acid segments used in the methods of the present invention, regardless of the length of the coding sequence itself, may be combined with other DNA sequences, such as promoters, enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a contiguous stretch of nucleotides identical to or complementary to SEQ ID NO:1, such as about 8, about 10 to about 14, or about 15 to about 20 nucleotides, and that are up to about 1,000,000, about 750,000, about 500,000, about 250,000, about 100,000, about 50,000, about 20,000, or about 10,000, or about 5,000 base pairs in length, with segments of about 3,000 being preferred in certain cases. In certain cases, nucleotide segments of a million bases or more, including chromosome sized pieces of DNA, are contemplated as being useful. DNA segments with total lengths of about 1,000, about 500, about 200, about 100 and about 50 base pairs in length (including all intermediate lengths) are also contemplated to be useful.

It will be readily understood that “intermediate lengths”, in these contexts, means any length between the quoted ranges, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200-500; 500-1,000; 1,000-2,000; 2,000-3,000; 3,000-5,000; 5,000-10,000 ranges, up to and including sequences of about 12,001, 12,002, 13,001, 13,002, 15,000, 20,000 and the like.

The various probes and primers designed around the disclosed nucleotide sequences of the present invention may be of any length. By assigning numeric values to a sequence, for example, the first residue is 1, the second residue is 2, etc., an algorithm defining all primers can be proposed:

n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

The nucleotides encoding the N5 gene of the present invention are also intended for use in the design of diagnostic and prognostic oligonucleotide probes and primers and for use in protein and peptide expression. When used in combination with nucleic acid amplification procedures, these probes and primers permit the rapid analysis of biopsy core specimens and other types of cancer cell specimens and samples for the ability of a cancer cell to express the N5 gene product(s) and also to quantitate or measure the levels of expression of the N5 gene product(s). The probes and primers can be used for in situ hybridization and/or in situ PCR™ analysis for prognosis of cancer by measuring the levels of expression of p84N5, which has been demonstrated herein to correlate to increasing the sensitization of a cancer cell to radiation and/or chemotherapy, therefore allowing for prescribing specific cancer treatment regimens based on the expression levels of N5 in a particular cancer.

It also will be understood that this invention is not limited to the use of particular nucleic acid and amino acid sequences of SEQ ID NO:1. Recombinant vectors and isolated DNA segments may therefore variously include these coding regions themselves, coding regions bearing selected alterations or modifications in the basic coding region, or they may encode larger polypeptides that nevertheless include such coding regions or may encode biologically functional equivalent proteins or peptides that have variant amino acids sequences.

The DNA segments useful in context of the present invention encompass biologically functional equivalent p84N5 proteins, polypeptides, and peptides. Such sequences may arise as a consequence of codon redundancy and functional equivalency that are known to occur naturally within nucleic acid sequences and the proteins thus encoded. Alternatively, functionally equivalent proteins, polypeptides or peptides may be created via the application of recombinant DNA technology, in which changes in the protein structure may be engineered, based on considerations of the properties of the amino acids being exchanged. Changes designed by man may be introduced, for example, through the application of site-directed mutagenesis techniques as discussed herein below, e.g., to introduce improvements to the antigenicity of the protein or to test mutants in order to examine p84N5 activity at the molecular level.

One also may prepare fusion proteins, polypeptides and peptides, e.g., where the p84N5 protein coding regions are aligned within the same expression unit with other proteins or peptides having desired functions, such as for purification or immunodetection purposes (e.g., proteins that may be purified by affinity chromatography and enzyme label coding regions, respectively).

Encompassed by the invention are DNA segments encoding relatively small peptides, such as, for example, peptides of from about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 40, about 45, to about 50 amino acids in length, and more preferably, of from about 15 to about 30 amino acids in length; as set forth in SEQ ID NO:2 and also larger polypeptides up to and including proteins corresponding to the full-length sequences set forth in SEQ ID NO:2.

IV. Quantitation of Levels of Expression of p84N5 Nucleic Acids

The inventors findings that expression levels of a p84N5 polypeptide/protein and/or mRNA increases the sensitivity of cancer cells to radiation and/or chemotherapeutic agents provides a prognostic method for prescribing optimal doses of radiation and/or chemotherapy treatments based on the levels of expression of p84N5 products in a particular cancer type, in a patient. Therefore, some embodiments of the invention concern measuring and/or quantitation and/or estimation of levels of p84N5 expression.

For quantitation of a nucleic acid, reverse transcription (RT) of RNA to cDNA followed by relative quantitative or semi-quantitative PCR™ (RT-PCR™) can be used to determine the relative concentrations of specific mRNA species in a series of total cell RNAs isolated from the cancer cells.

By determining that the concentration of a specific mRNA species varies, it is shown that the gene encoding the specific mRNA species is expressed at different levels in different types of cancers.

In PCR™, the number of molecules of the amplified target DNA increase by a factor approaching two with every cycle of the reaction until some reagent becomes limiting. Thereafter, the rate of amplification becomes increasingly diminished until there is not an increase in the amplified target between cycles. If one plots a graph on which the cycle number is on the X axis and the log of the concentration of the amplified target DNA is on the Y axis, one observes that a curved line of characteristic shape is formed by connecting the plotted points.

Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ is directly proportional to the starting concentration of the target before the PCR™ was begun. By determining the concentration of the PCR™ products of the target DNA in PCR™ reactions that have completed the same number of cycles and are in their linear ranges, it is possible to determine the relative concentrations of the specific target sequence in the original DNA mixture.

If the DNA mixtures are cDNAs synthesized from RNAs isolated from different cells, the relative abundances of the specific mRNA from which the target sequence was derived can be determined for the respective tissues or cells. This direct proportionality between the concentration of the PCR™ products and the relative mRNA abundances is only true in the linear range portion of the PCR™ reaction.

The final concentration of the target DNA in the plateau portion of the curve is determined by the availability of reagents in the reaction mix and is independent the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ study to successfully determine the relative abundances of a particular mRNA species is that relative concentrations of the amplifiable cDNAs must be normalized to some independent standard. The goal of an RT-PCR™ study is to determine the abundance of a particular mRNA species relative to the average abundance of all mRNA species in the sample. In such studies, mRNAs for β-actin, asparagine synthetase and lipocortin II may be used as external and internal standards to which the relative abundance of other mRNAs are compared.

Most protocols for competitive PCR™ utilize internal PCR™ internal standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The discussion above describes the theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target).

Both of the foregoing problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies are available that use a more conventional relative quantitative RT-PCR™ with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This is very important since this assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR m assays can be superior to those derived from the relative quantitative RT-PCR™ with an internal standard.

One reason for this is that without the internal standard/competitor, all of the reagents can be converted into a single PCR™ product in the linear range of the amplification curve, increasing the sensitivity of the assay. Another reason is that with only one PCR™ product, display of the product on an electrophoretic gel or some other display method becomes less complex, has less background and is easier to interpret.

V Genetic Constructs

Throughout this application, the term “expression construct” is meant to include any type of genetic construct containing a nucleic acid coding for gene products, such as p84N5, in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding genes of interest.

(i) Promoters

The nucleic acid encoding a gene product is under transcriptional control of a promoter. A “promoter” refers to a DNA sequence recognized by the machinery of the cell, or introduced machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can pennit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constituitively expressed from one vector, whereas the ecdysone-responsive promoter which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A.

Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constituitively on. In the Tet-On™ system, the tetracycline repressor is not wild-type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constituitively on.

In some circumstances, it may be desirable to regulate expression of a transgene in a gene therapy vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the insulin, elastin, Amylase, pdr-1, pdx-1, or glucokinase promoters may be used to target gene expression to pancreatic cancers. Promoters such as PSA, probasin, prostatic acid phosphatase or prostate-specific glandular kallikrein (hK2) may be used to target gene expression in the prostate. Similarly, the following promoters may be used to target gene expression in other tissues (Table 1).

TABLE 1
Tissue Specific Promoters

| Tissue | Promoter |
|---|---|
| Pancreas | insulin |
| | elastin |
| | amylase |
| | pdr-1 pdx-1 |
| | glucokinase |
| Liver | albumin PEPCK |
| | HBV enhancer |
| | alpha fetoprotein |
| | apolipoprotein C |
| | alpha-1 antitrypsin |
| | vitellogenin, NF-AB |
| | Transthyretin |
| Skeletal muscle | myosin H chain |
| | muscle creatine kinase |
| | dystrophin |
| | calpain p94 |
| | skeletal alpha-actin |
| | fast troponin 1 |
| Skin | keratin K6 |
| | keratin K1 |
| Lung | CFTR |
| | human cytokeratin 18 (K18) |
| | pulmonary surfactant proteins A, B and C |
| | CC-10 |
| | P1 |
| Smooth muscle | sm22 alpha |
| | SM-alpha-actin |
| Endothelium | endothelin-1 |
| | E-selectin |
| | von Willebrand factor |
| | TIE (Korhonen et al., 1995) |
| | KDR/flk-1 |
| Melanocytes | tyrosinase |
| Adipose tissue | lipoprotein lipase (Zechner et al., 1988) |
| | adipsin (Spiegelman et al., 1989) |
| | acetyl-CoA carboxylase (Pape and Kim, 1989) |
| | glycerophosphate dehydrogenase (Dani et al., 1989) |
| | adipocyte P2 (Hunt et al., 1986) |
| Blood | β-globin |

In certain indications, it may be desirable to activate transcription at specific times after administration of the gene therapy vector. This may be done with such promoters as those that are hormone or cytokine regulatable. For example in gene therapy applications where the indication is a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful in the present invention. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-I acid glycoprotein (Prowse and Baumann, 1988), alpha-I antitypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 antichymotrypsin.

It is envisioned that cell cycle regulatable promoters may be useful in the present invention. For example, in a bi-cistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2F1, p53 and BRCA1 could be used.

Tumor specific promoters such as osteocalcin, hypoxia-responsive element (HRE), MAGE-4, CEA, alpha-fetoprotein, GRP78/BiP and tyrosinase also may be used to regulate gene expression in tumor cells. Other promoters that could be used according to the present invention include Lac-regulatable, chemotherapy inducible (e.g. MDR), and heat (hypeithemlia) inducible promoters, Radiation-inducible (e.g., EGR (Joki et al., 1995)), Alpha-inhibin, RNA pol III tRNA met and other amino acid promoters, Ul snRNA (Bartlett et al., 1996), MC-1, PGK, -actin and alpha-globin. Many other promoters that may be useful are listed in Walther and Stein (1996).

It is envisioned that any of the above promoters alone or in combination with another may be useful according to the present invention depending on the action desired. In addition, this list of promoters is should not be construed to be exhaustive or limiting, those of skill in the art will know of other promoters that may be used in conjunction with the promoters and methods disclosed herein.

(ii) Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters additional to the tissue specific promoters listed above, cellular promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2

Enhancers
Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94
and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 3

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone | Thyroid Hormone |
| α Gene | |
| Insulin E Box | Glucose |

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

(iii) Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

VI. Antisense Constructs

The term “antisense nucleic acid” is intended to refer to the oligonucleotides complementary to the base sequences of DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the terms “complementary” or “antisense sequences” mean nucleic acid sequences that are substantially complementary over their entire length and have very few base mismatches. For example, nucleic acid sequences of fifteen bases in length may be tenned complementary when they have a complementary nucleotide at thirteen or fourteen positions with only single or double mismatches. Naturally, nucleic acid sequences which are “completely complementary” will be nucleic acid sequences which are entirely complementary throughout their entire length and have no base mismatches.

While all or part of the gene sequence may be employed in the context of antisense construction, statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

VII. Ribozyme Constructs

As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

VIII. Methods of Gene Transfer

In order to mediate the effect of transgene expression in a cell, it will be necessary to transfer the therapeutic expression constructs of the present invention into a cell. This section provides a discussion of methods and compositions of viral production and viral gene transfer, as well as non-viral gene transfer methods.

(i) Viral Vector-Mediated Transfer

The p84N5 genes are incorporated into a viral infectious particle to mediate gene transfer to a cell. Additional expression constructs encoding other therapeutic agents as described herein may also be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Viral vectors contemplated as useful include both replication-defective as well as replication-competent viral vectors (Matsubara et al., 2001; Rodrigez et al., 1997; Yu et al., 1999; Heise and Kim, 2000; Kim, 2000; Galanis et al., 2001). Though adenovirus is exemplified, the present methods may be advantageously employed with other viral or non-viral vectors, as discussed below.

Adenovirus. Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage ? DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the EIA (194-358 bp) region of the genome grew poorly even in a cell line that complemented the early (EIA) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

The use of replication-competent viral vectors is also contemplated (Matsubara et al., 2001; Rodrigez et al., 1997; Yu et al., 1999; Heise and Kim, 2000; Kirn, 2000; Galanis et al., 2001).

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and T components is constructed (Mann et al., 1983). When a recombinant plasmid containing a human cDNA, together with the retroviral LTR and T sequences is introduced into this cell line (by calcium phosphate precipitation for example), the T sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

Adeno-Associated Virus. AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p20%, which contains a modified AAV genome (Samulski et al. 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor 1×gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

Other Viral Vectors. Other viral vectors may be employed as expression constructs in the present invention. Vectors derived from viruses such as vaccinia virus (Ridgeway, 1988; Baichwal and Sugden, 1986; Coupar et al., 1988) and hepatitus B viruses have also been developed and are useful in the present invention. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al., recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

(ii) Non-Viral Transfer

DNA constructs useful in the context of the present invention are generally delivered to a cell, in certain situations, the nucleic acid to be transferred is non-infectious, and can be transferred using non-viral methods.

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nicleic acid encoding the therapeutic gene may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

IX. p84N5 Expression and Purification Systems

Both cDNA and genomic sequences are suitable for eukaryotic expression, as the host cell will generally process the genomic transcripts to yield functional mRNA for translation into protein. For prokaryotic expression, cDNA sequences are preferred. Generally speaking, it may be more convenient to employ as the recombinant gene a cDNA version of the gene, such as that shown in SEQ ID NO: 1. It is believed that the use of a cDNA version will provide advantages in that the size of the gene will generally be much smaller and more readily employed to transfect the targeted cell than will a genomic gene, which will typically be up to an order of magnitude or more larger than the cDNA gene. However, it is contemplated that a genomic version of a particular gene may be employed where desired.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

It is proposed that p84N5 proteins, polypeptides or peptides may be co-expressed with other selected proteins, wherein the proteins may be co-expressed in the same cell or p84N5 gene may be provided to a cell that already has another selected protein. Co-expression may be achieved by co-transfecting the cell with two distinct recombinant vectors, each bearing a copy of either of the respective DNA. Alternatively, a single recombinant vector may be constructed to include the coding regions for both of the proteins, which could then be expressed in cells transfected with the single vector. In either event, the term “co-expression” herein refers to the expression of both the p84N5 gene and the other selected protein in the same recombinant cell.

As used herein, the terms “engineered” and “recombinant” cells or host cells are intended to refer to a cell into which an exogenous DNA segment or gene, such as a cDNA or gene encoding an p84N5 protein has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant p84N5 protein, polypeptide or peptide, whether mutant or wild-type, in accordance with the present invention one would prepare an expression vector that comprises a wild-type, or mutant p84N5 protein-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein. This is the meaning of “recombinant expression” in this context.

Many standard techniques are available to construct expression vectors containing the appropriate nucleic acids and transcriptional/translational control sequences in order to achieve protein, polypeptide or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as E. coli LE392.

Further useful vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The following details concerning recombinant protein production in bacterial cells, such as E. coli, are provided by way of exemplary information on recombinant protein production in general, the adaptation of which to a particular recombinant expression system will be known to those of skill in the art.

Bacterial cells, for example, E. coli, containing the expression vector are grown in any of a number of suitable media, for example, LB. The expression of the recombinant protein may be induced, e.g., by adding IPTG to the media or by switching incubation to a higher temperature. After culturing the bacteria for a further period, generally of between 2 and 24 hours, the cells are collected by centrifugation and washed to remove residual media.

The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g., 8M) or chaotropic agents such as guanidine hydrochloride in the presence of reducing agents, such as P-mercaptoethanol or DTT (dithiothreitol).

Under some circumstances, it may be advantageous to incubate the protein for several hours under conditions suitable for the protein to undergo a refolding process into a conformation which more closely resembles that of the native protein. Such conditions generally include low protein concentrations, less than 500 mg/ml, low levels of reducing agent, concentrations of urea less than 2 M and often the presence of reagents such as a mixture of reduced and oxidized glutathione which facilitate the interchange of disulfide bonds within the protein molecule.

The refolding process can be monitored, for example, by SDS-PAGE, or with antibodies specific for the native molecule (which can be obtained from animals vaccinated with the native molecule or smaller quantities of recombinant protein). Following refolding, the protein can then be purified further and separated from the refolding mixture by chromatography on any of several supports including ion exchange resins, gel permeation resins or on a variety of affinity columns.

For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more p84N5 protein, polypeptide or peptide coding sequences.

In a useful insect system, Autograph californica nuclear polyhedrosis virus (AcNPV) is used as a vector to express foreign genes. The virus grows in Spodoptera frugiperda cells. The p84N5 protein, polypeptide or peptide coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051, Smith, incorporated herein by reference).

Examples of useful mammalian host cell lines are VERO and HeLa cells, Chinese hamster ovary (CHO) cell lines, WI38, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein.

Different host cells have characteristic and specific mechanisms for the post-translational processing and modification of proteins. Appropriate cells lines or host systems can be chosen to ensure the correct modification and processing of the foreign protein expressed.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the p84N5, provided such control sequences are compatible with the host cell systems.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1, E3, or E4) will result in a recombinant virus that is viable and capable of expressing p84N5 proteins, polypeptides or peptides in infected hosts.

Specific initiation signals may also be required for efficient translation of p84N5 protein, polypeptide or peptide coding sequences. These signals include the ATG initiation codon and adjacent sequences. Exogenous translational control signals, including the ATG initiation codon, may additionally need to be provided. One of ordinary skill in the art would readily be capable of determining this and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements and transcription terminators.

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

For long-term, high-yield production of a recombinant p84N5 protein, polypeptide or peptide, stable expression is preferred. For example, cell lines that stably express constructs encoding an p84N5 protein, polypeptide or peptide may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including, but not limited to, the herpes simplex virus thymidine kinase (tk), hypoxanthine-guanine phosphoribosyltransferase (hgprt) and adenine phosphoribosyltransferase (aprt) genes, in tk⁻, hgprt⁻ or aprt⁻ cells, respectively. Also, antimetabolite resistance can be used as the basis of selection for dihydrofolate reductase (dhfr), that confers resistance to methotrexate; gpt, that confers resistance to mycophenolic acid; neomycin (neo), that confers resistance to the aminoglycoside G-418; and hygromycin (hygro), that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

It is contemplated that the p84N5 proteins, polypeptides or peptides used in context of the cancers therapies described herein may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein or peptide in comparison to the level in natural cells is indicative of overexpression, as is a relative abundance of the specific protein in relation to the other proteins produced by the host cell and, e.g., visible on a gel.

Purification techniques may find use in the current invention, for example, in the purification of the p84N5 protein. Purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; and isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

X. Antibodies

(i) Antibody Generation

It will be understood that polyclonal or monoclonal antibodies specific for a p84N5 polypeptide/protein will have utilities in several applications. These include the production of prognostic kits for use in detecting the levels of expression of p84N5 in a particular cancer and prescribing an optimal dose of a cancer therapeutic regimen comprising radiation and/or chemotherapeutic drugs with or without the gene therapeutic regimens described herein.

Thus the invention further provides antibodies specific for the proteins, polypeptides or peptides of p84N5, encoded by the nucleic acid segments disclosed herein and their equivalents. Means for preparing and characterizing antibodies are well known in the art (Harland and Lane, 1988; incorporated herein by reference). Antibodies to p84N5 peptides or protein have already been generated using such standard techniques.

The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal.

A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate mAbs.

For production of rabbit polyclonal antibodies, the animal can be bled through an ear vein or alternatively by cardiac puncture. The procured blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix or protein A followed by antigen (peptide) affinity column for purification.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified p84N5 protein, polypeptide or peptide. The immunizing composition is administered in a manner effective to stimulate antibody producing cells.

The methods for generating mAbs generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the mAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Agl4, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine mycloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific mAb produced by the fused cell hybrid. The body fluids of the animal, such as serum or ascites fluid, can then be tapped to provide mAbs in high concentration.

The individual cell lines could also be cultured in vitro, where the mAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the mAbs of the invention can be obtained from the purified mAbs by methods which include digestion with enzymes, such as pepsin or papain, and/or by cleavage of disulfide bonds by chemical reduction. Alternatively, mAb fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized mAbs are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens such antibodies are generally useful for in vivo therapeutic applications. This strategy reduces the host response to the foreign antibody and allows selection of the human effector functions.

The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope.

Other U.S. patents, each incorporated herein by reference, that teach the production of antibodies useful in the present invention include U.S. Pat. No. 5,565,332, which describes the production of chimeric antibodies using a combinatorial approach; 4,816,567 which describes recombinant immunoglobin preparations and 4,867,973 which describes antibody-therapeutic agent conjugates.

U.S. Pat. No. 5,565,332 describes methods for the production of antibodies, or antibody fragments, which have the same binding specificity as a parent antibody but which have increased human characteristics. Humanized antibodies may be obtained by chain shuffling, perhaps using phage display technology, in as much as such methods will be useful in the present invention the entire text of U.S. Pat. No. 5,565,332 is incorporated herein by reference. Human antibodies may also be produced by transforming B cells with EBV and subsequent cloning of secretors as described by Hoon et al., (1993).

The invention further encompasses anti-p84N5 antibodies and antibody-based compositions, such as antibody conjugates and immunotoxins, that bind to the same antigens and/or epitopes as the antibodies disclosed herein (e.g., those raised to the peptides of SEQ ID NO:2). Such antibodies may be of the polyclonal or monoclonal type, with monoclonals being generally preferred.

The identification of an antibody that binds to a p84N5 polypeptide or an epitope thereof, in substantially the same manner as an antibody of the invention is a fairly straightforward matter. This can be readily determined using any one of variety of immunological screening assays in which antibody competition can be assessed.

For example, where the test antibodies to be examined are obtained from different source animals, or are even of a different isotype, a simple competition assay may be employed in which the control and test antibodies are premixed and then applied to an antigen composition. By “antigen composition” is meant any composition that contains a p84N5 polypeptide/protein or peptide. Thus, protocols based upon ELISAs and Western blotting are suitable for use in such simple competition studies.

In such embodiments, one would pre-mix the control antibodies with varying amounts of the test antibodies (e.g., 1:1, 1:10 and 1:100) for a period of time prior to applying to an antigen composition, such as an antigen-coated well of an ELISA plate or an antigen adsorbed to a membrane (as in dot blots and Western blots). By using species or isotype secondary antibodies one will be able to detect only the bound control antibodies, the binding of which will be reduced by the presence of a test antibody that recognizes the same epitope/antigen.

(ii) Antibody Conjugates

Antibody conjugates in which a p84N5 antibody is linked to a detectable label form further aspects of the invention. Prognostic/diagnostic antibody conjugates may be used in vitro prognostic assays, as in a variety of immunoassays.

Certain antibody conjugates include those, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Radioactively labeled mAbs of the present invention may be produced according to well-known methods in the art. For instance, mAbs can be iodinated by contact with sodium or potassium iodide and a chemical oxidizing agent such as sodium hypochlorite, or an enzymatic oxidizing agent, such as lactoperoxidase. MAbs according to the invention may be labeled with technetium-^{99m} by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) and ethylene diaminetetracetic acid (EDTA). Fluorescent labels include rhodamine, fluorescein isothiocyanate and renographin.

XI. Immunological Detection

The p84N5 antibodies of the invention are useful in various prognostic applications connected with the analysis of cancer and the determination of what type and dose of radio or chemotherapeutic regimens are suitable based on the levels of expression of p84N5 in a cancer.

In still further embodiments, the present invention thus concerns immunodetection methods for binding, purifying, removing, quantifying or otherwise generally detecting p84N5. The steps of various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, a cancer cell sample will be contacted with an antibody and then the amount of immune complexes formed under the specific conditions will be detect or quantified.

In terms of antigen detection, the biological sample analyzed may be any sample such as a cancer of the breast, gastric, colon, pancreas, renal, ovarian, lung, prostate, hepatic, brain, bone, lymph node or bone marrow tissue section or specimen, a homogenized tissue extract, an isolated cell, a cell membrane preparation, separated or purified forms of any of the above protein-containing compositions, or even any biological fluid that comes into contact with cancer tissues, including blood, lymphatic fluid, seminal fluid and urine.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The antibody employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the prognosis of cancer as the inventors have demonstrated herein an increased radiation sensitivity and/or chemotherapeutic sensitivity of cancers that express higher levels of p84N5. However, these embodiments also have applications to non-clinical samples, such as in the titering of antigen or antibody samples, in the selection of hybridomas, and the like.

The antibodies described herein also may be used in conjunction with both fresh-frozen and formalin-fixed, paraffin-embedded tissue blocks prepared from study by immunohistochemistry (1HC). For example, each tissue block consists of 50 mg of residual “pulverized” tumor. The method of preparing tissue blocks from these particulate specimens has been successfully used in previous IHC studies of various prognostic factors, e.g., in breast, and is well known to those of skill in the art (Brown et al., 1990; Abbondanzo et al., 1999; Allred et al., 1990).

Briefly, frozen-sections may be prepared by rehydrating 50 ng of frozen “pulverized” tumor at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 remarkably intact tumor cells.

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

Fluorescent activated cell sorting, flow cytometry or flow microfluorometry provides the means of scanning individual cells for the presence of an antigen, such as p84N5. The method employs instrumentation that is capable of activating, and detecting the excitation emissions of labeled cells in a liquid medium.

FACS is unique in its ability to provide a rapid, reliable, quantitative, and multiparameter analysis on either living or fixed cells. The cancer antibodies of the present invention provide a useful tool for the analysis and quantitation of antigenic cancer markers of individual cells.

Cells would generally be obtained by biopsy, single cell suspension in blood or culture. FACS analyses would probably be most useful when desiring to analyze a number of cancer antigens at a given time, e.g., to follow an antigen profile during disease progression.

XII. Pharmaceuticals

In a particular aspect, the present invention provides methods for the treatment of various pancreatic cancers such as, but not limited to, ductal adenocarcinoma, mucinous cystadenocarcinoma, acinar carcinoma, unclassified large cell carcinoma, small cell carcinoma, pancreatoblastoma, intraductal papillary neoplasm, mucinous cystadnoma, and papillary cystic neoplasm and ovarian cancers such as, but not limited to, serous, mucinous, endometrioid, clear cell mesonephroid, Brenner, or mixed epithelial cancer. In some embodiments, the treatment methods will involve treating an afflicated individual with an effective amount of a viral particle, as described above, containing a gene encoding a p84N5 death domain. Alternatively, treatment methods will involve treating an individual with an effective amount of a p84N5 protein composition. An effective amount is described, generally, as that amount sufficient to detectably and repeatedly to induce apoptosis, inhibit cell division, inhibit metastatic potential, reduce tumor burden, increase sensitivity to chemotherapy or radiotherapy, kill a cancer cell, inhibit the growth of a cancer cell, or induce tumor regression.

To kill cells, inhibit cell growth, inhibit metastasis, decrease tumor size and otherwise reverse or reduce the malignant phenotype of tumor cells, using the methods and compositions of the present invention, one would generally contact a “target” cell with the therapeutic viral or protein composition. This may be combined with compositions comprising other agents effective in the treatment of cancer. These compositions would be provided in a combined amount effective to kill or inhibit proliferation of the cell.

Administration of the therapeutic viral or protein composition of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described treatments.

Where clinical application of a composition is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Depending on the particular cancer to be, administration of therapeutic compositions according to the present invention will be via any common route so long as the target tissue is available via that route. This includes oral, nasal, buccal, rectal, vaginal or topical. Topical administration would be particularly advantageous for treatment of skin cancers. Alternatively, administration will be by orthotopic, intradenmal, subcutaneous, intramuscular, intraperitoneal or intravenous injection. Such compositions would normally be administered as pharmaceutically acceptable compositions that include physiologically acceptable carriers, buffers or other excipients.

In certain embodiments, ex vivo therapies also are contemplated. Ex vivo therapies involve the removal, from a patient, of target cells. The cells are treated outside the patient's body and then returned. One example of ex vivo therapy would involve a variation of autologous bone marrow transplant. Many times, ABMT fails because some cancer cells are present in the withdrawn bone marrow, and return of the bone marrow to the treated patient results in repopulation of the patient with cancer cells. In one embodiment, however, the withdrawn bone marrow cells could be treated while outside the patient with an viral particle that targets and kills the cancer cell. Once the bone marrow cells are “purged,” they can be reintroduced into the patient.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

Preferably, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

One of the preferred embodiments of the present invention involves the use of viral vectors to deliver therapeutic genes to cancer cells. Alternatively, another embodiment of the present invention involves the use of therapeutic protein compositions. Target cancer cells include cancers of the pancreas, lung, brain, prostate, kidney, liver, ovary, breast, skin, stomach, esophagus, head and neck, testicles, colon, cervix, lymphatic system and blood. Of particular interest are pancreatic and ovarian cancers.

According to the present invention, one may treat the cancer by directly injection a tumor with the viral vector or protein composition. Alternatively, the tumor may be infused or perfused with the vector or protein using any suitable delivery vehicle. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles or protein may advantageously be contacted by administering multiple injections to the tumor, spaced at approximately 1 cm intervals.

In certain embodiments, the tumor being treated may not, at least initially, be resectable. Treatments with therapeutic viral constructs or protein compositions may increase the resectability of the tumor due to shrinkage at the margins or by elimination of certain particularly invasive portions. Following treatments, resection may be possible. Additional viral or protein treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

(i) Pharmaceutical Formulations

Aqueous compositions of the present invention comprise an effective amount of the p84N5 protein or recombinant viral vector encoding p84N5 dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards. The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an p84N5 polypeptide or p84N5 encoding gene composition as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An p84N5 polypeptide or p84N5 polypeptide encoding gene composition can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In terms of using peptide therapeutics as active ingredients, the technology of U.S. Pat. Nos. 4,608,251; 4,601,903; 4,599,231; 4,599,230; 4,596,792; and 4,578,770, each incorporated herein by reference, may be used.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The active p84N5 polypeptides or agents may be formulated within a therapeutic mixture to comprise about 0.0001 to 1.0 milligrams, or about 0.001 to 0.1 milligrams, or about 0.1 to 1.0 or even about 10 milligrams per dose or so. In preferred embodiments, the active p84N5 polypeptides or agents are formulated within a therapeutic mixture to comprise about 0.001 to about 1 milligram. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used, including cremes.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5.

In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository may also be used.

Suppositories are solid dosage forms of various weights and shapes, usually medicated, for insertion into the rectum, vagina or the urethra. After insertion, suppositories soften, melt or dissolve in the cavity fluids.

In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

XIII. Combination Cancer Therapies

In order to further enhance the efficacy of the gene therapy provided by the invention, combination therapies are contemplated. Thus, a second therapeutic agent in addition to the p84N5 based therapy may be used. The second therapeutic agent may be a chemotherapeutic agent, a radiotherapeutic agent, a gene therapeutic agent, a protein/peptide/polypeptide therapeutic agent, another immunotherapeutic agent, etc. Such agents are well known in the art.

“Effective amount” is defined as an amount of the agent that will decrease, reduce, inhibit or otherwise abrogate the growth of a cancer cell, arrest-cell growth, induce apoptosis, inhibit metastasis, induce tumor necrosis, kill cells or induce cytotoxicity in cells.

The administration of the second therapeutic agent may precede or follow the therapy using a p84N5 encoding nucleic acid or protein product by intervals ranging from minutes to days to weeks. In embodiments where the second therapeutic agent and a p84N5 encoding nucleic acid or protein product are administered together, one would generally ensure that a significant period of time did not expire between the time of each delivery. In such instances, it is contemplated that one would administer to a patient both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the second therapeutic agent and the p84N5 encoding nucleic acid or protein product will be required to achieve complete cancer cure. Various combinations may be employed, where the second therapeutic agent is “A” and the p84N5 encoding nucleic acid or protein product is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations also are contemplated. The exact dosages and regimens of each agent can be suitable altered by those of ordinary skill in the art.

Provided below is a description of some other agents effective in the treatment of cancer.

(i) Radiotherapeutic Agents

In some tumor cell lines, levels of endogenous p84N5, were found to correlate to the sensitivity of cells to ionizing radiation, indicating that N5 gene therapy restores and/or enhances sensitivity of tumor cells to genotoxic agents. Therefore, additional therapy with radiotherapeutic agents and factors including radiation and waves that induce DNA damage for example, y-irradiation, X-rays, UV-irradiation, microwaves, electronic emissions, radioisotopes, and the like are contemplated. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

(ii) Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 1, or 12 months. These treatments may be of varying dosages as well.

(iii) Chemotherapeutic Agents

Agents that damage DNA are chemotherapeutics. These can be, for example, agents that directly cross-link DNA, agents that intercalate into DNA, and agents that lead to chromosomal and mitotic aberrations by affecting nucleic acid synthesis. Agents that directly cross-link nucleic acids, specifically DNA, are envisaged and are exemplified by cisplatin, and other DNA alkylating agents. Agents that damage DNA also include compounds that interfere with DNA replication, mitosis, and chromosomal segregation.

Some examples of chemotherapeutic agents include antibiotic chemotherapeutics such as, Doxorubicin, Daunorubicin, Mitomycin (also known as mutamycin and/or mitomycin-C), Actinomycin D (Dactinomycin), Bleomycin, Plicomycin,. Plant alkaloids such as Taxol, Vincristine, Vinblastine. Miscellaneous agents such as Cisplatin, VP16, Tumor Necrosis Factor. Alkylating Agents such as Carmustine, Melphalan (also-knowna as alkeran, L-phenylalanine mustard, phenylalanine mustard, L-PAM, or L-sarcolysin, is a phenylalanine derivative of nitrogen mustard), Cyclophosphamide, Chlorambucil, Busulfan (also known as myleran), Lomustine. And other agents for example, Cisplatin (CDDP), Carboplatin, Procarbazine, Mechlorethamine, Camptothecin, Ifosfamide, Nitrosurea, Etoposide (VP16), Tamoxifen, Raloxifene, Estrogen Receptor Binding Agents, Gemcitabien, Navelbine, Farnesyl-protein transferase inhibitors, Transplatinum, 5-Fluorouracil, and Methotrexate, Temazolomide (an aqueous form of DTIC), or any analog or derivative variant of the foregoing.

(iv) Immunotherapy

Immunotherapeutics may be used in conjunction with the therapy using a p84N5 encoding nucleic acid or protein product. Immunotherapeutics, generally, rely on the use of immune effector cells and molecules to target and destroy cancer cells. The immune effector may be, for example, another antibody specific for some other marker on the surface of a tumor cell. This antibody in itself may serve as an effector of therapy or it may recruit other cells to actually effect cell killing. This antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. Immunotherapy could be used as part of a combined therapy, in conjunction with the anti-CD26 antibody-based therapy.

The general approach for combined therapy is discussed below. In one aspect the immunotherapy can be used to target a tumor cell. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p5. Alternate immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with the anti-CD26 antibody-based therapy of this invention will enhance anti-tumor effects.

(a) Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

(b) Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996).

(c) Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

(v) Gene Therapy

In yet another embodiment, another gene therapy in conjunction with the gene therapy using a p84N5 encoding nucleic acid or protein product described in the invention are contemplated. A variety of nucleic acids and proteins encoded by nucleic acids are encompassed within the invention, some of which are described below. Table 4 lists various genes that may be targeted for gene therapy of some form in combination with the present invention.

TABLE 4

| Gene | Source | Human Disease | Function |
|---|---|---|---|
| Growth Factors | | | |
| HST/KS | Transfection | | FGF family member |
| INT-2 | MMTV promoter | | FGF family member |
| | Insertion | | |
| INTI/WNTI | MMTV promoter | | Factor-like |
| | Insertion | | |
| SIS | Simian sarcoma virus | | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | squamous cell | Amphiregulin/ |
| | insertion; amplified | cancer; glioblastoma | Hetacellulin receptor |
| | human tumors | | |
| ERBB-2/NEU/HER-2 | Transfected from rat | Amplified breast, | Regulated by NDF/ |
| | Glioblastomas | ovarian, gastric cancers | Heregulin and EGF- |
| | | | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid cancer; | Orphan receptor Tyr |
| | mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |

NONRECEPTOR TYROSINE KINASES

| | | | |
|---|---|---|---|
| ABI | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine leukemia | | Src family; T-cell |
| | virus) promoter | | signaling; interacts |
| | insertion | | CD4/CD8 T-cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE¹

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gh homogue |
| | | | CI to antagonize |
| | | | hedgehog pathway |
| TAN-1 Notch | Translocation | T-ALI. | Signaling? |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor 5H2/5H3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N-RAS | HaRat SV; K1 RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | 1 transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NT-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| MDA-7 | Fragile site 3p14.2 | Lung carcinoma | Histidine triad-related |
| | | | diadenosine 5′,3′′′′- |
| | | | tetraphosphate |
| | | | asymmetric hydrolase |
| hML1/MutL | | HNPCC | Mismatch repair; MutL |
| | | | Homologue |
| hMSH2/MutS | | HNPCC | Mismatch repair; MutS |
| | | | Homologue |
| hPMS1 | | HNPCC | Mismatch repair; MutL |
| | | | Homologue |
| hPMS2 | | HNPCC | Mismatch repair; MutL |
| | | | Homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

(vi) Other Agents

It is contemplated that other agents may be used in combination with the present invention to improve the therapeutic efficacy of treatment. One form of therapy for use in conjunction with chemotherapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen and this often reduces the risk of metastases.

The terms “contacted” and “exposed,” when applied to a cell, are used herein to describe the process by which a therapeutic construct or protein and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

XIV. Prognostic Applications

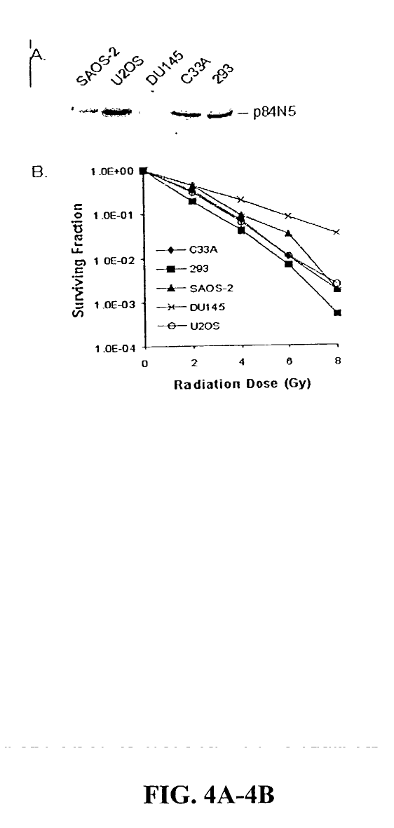

The present inventors have shown that the expression levels of p84N5 is different in different types of cancer cells and cell lines. In addition, they have also determined a correlation between the sensitivity of a cancer cell to radiation treatments and the levels of expression of p84N5 polypeptides/protein and/or mRNA (FIG. 4A and FIG. 4B). Therefore, cells that express more p84N5 are more sensitive to radiation. The inventors contemplate performing similar experiments with chemotherapeutic agents to determine if the expression of p84N5 increases sensitivity to chemotherapeutic agents as well. Based on these studies, the present inventors contemplate that the expression level of a p84N5 polypeptide/protein and/or mRNA in a cancer cell, will be useful to effectively predict the efficacy of a radiation-based and/or a chemotherapeutic-based cancer therapeutic regimen for that particular cancer.

Thus, evaluation of the level of expression of a p84N5 polypeptide/protein and/or mRNA in the cancer tissues or cancer cells of a patient will be useful in determining whether that patient's cancer will be responsive to a particular cancer therapeutic regimen. The present invention therefore, provides a prognostic method which allows the determination of the need for specific cancer therapeutic regimens based on the expression profile of p84N5 in an individual patient.

The expression levels of p84N5 will also be useful in monitoring the effectiveness of a treatment regimen, such as a gene therapeutic regimen of the present invention, alone or in conjunction with other cancer therapies as described above. Again, in such a situation the level of expression of p84N5 will be used to effectively determine and adjust the dosage of a radiation and/or chemotherapeutic combination regimen. In any event, the methods of the present invention will assist physicians in determining optimal treatment courses and doses for individuals with different tumors of varying malignancy based on the levels of expression of p84N5 products in such tumors.

As described herein, the amount of a p84N5 polypeptide/protein and/or mRNA present within a biological sample or specimen, such as a tissue, a cell(s), blood or serum or plasma sample, any other biological fluid, a biopsy, needle biopsy cores, surgical resection samples, lymph node tissue, or any other clinical sample may be determined by means of a molecular biological assay to determine the level of a nucleic acid that encodes such a polypeptide, or by means of a polypeptide/protein detection assay such as a western blot, or even by means of an immunoassay may be detected and measured or quantified. Such detection and measuring/quantification methods are known to one of skill in the art.

In certain embodiments, nucleic acids or polypeptides would be extracted from these samples and amplified or detected as described above. Some embodiments would utilize kits containing pre-selected primer pairs or hybridization probes or antibodies. The amplified nucleic acids or polypeptide would be tested for the presence of a p84N5 polypeptide/protein and/or mRNA by any of the detection methods described herein or other suitable methods known in the art.

In other embodiments, sample/specimen extracts containing a p84N5 polypeptide/protein and/or mRNA would be extracted from a sample and subjected to an immunoassay. Immunoassays of tissue sections are also possible. Immunoassays that are contemplated useful are well known to one of skill in the art. Kits containing the antibodies to N5 polypeptides would be useful.

In terms of analyzing tissue samples, irrespective of the manner in which the level of a p84N5 polypeptide/protein and/or mRNA is determined, the prognostic evaluation may generally require the amount of the p84N5 product in the tissue sample to be compared to the amount in normal cells, in other patients and/or amounts at an earlier stage of treatment of the same patient. Comparing the varying levels will allow the characteristics of the particular cancer to be more precisely defined and therefore allow for prescribing a tailor made cancer treatment regimen to a patient.

Thus, it is contemplated that the levels of a p84N5 polypeptide/protein and/or mRNA detected would be compared with statistically valid groups of metastatic, non-metastatic malignant, benign or normal tissue samples; and/or with earlier p84N5 levels in the same patient. The diagnosis and prognosis of the individual patient would be determined by comparison with such groups.

For example, in a case where there is an increase in the expression level of a p84N5 product after a gene therapeutic intervention as described herein, the physician can decrease the amount of radiation and/or chemotherapy as the cancer cells now are rendered more sensitive to the radiation and/or chemotherapy due to increased p84N5 polypeptides/proteins in the cancer cells. In other examples, where a gene therapy is not performed, one may simply detect the expression level of p84N5 products and if a cancer does not express p84N5 or expresses only low levels of p84N5, the patient can be selected for either a gene therapeutic intervention as described in the present invention and/or allow the physician to prescribe a higher radiation and/or chemotherapeutic dosage as the cancer is likely not very sensitive to lower levels.

If desired, the cancer prognostic methods of the present invention may be readily combined with other methods in order to provide an even more reliable indication of prognosis. Various markers of cancer have been proposed to be correlated with metastasis and malignancy. They are generally classified as cytological, protein or nucleic acid markers. Any one or more of such methods may thus be combined with those of this invention in order to provide a multi-marker prognostic test. Some examples of cancer markers include, cytological markers include such things as “nuclear roundedness” and cell ploidy; protein markers include prostate specific antigen (PSA) and CA125; nucleic acid markers such as amplification of Her2/neu, point mutations in the p53 or Ras genes, and changes in the sizes of triplet repeat segments of particular chromosomes.

Combination of the present techniques with one or more other diagnostic or prognostic techniques or markers is certainly contemplated. In that many cancers are multifactorial, the use of more than one method or marker is often highly desirable.

XV. Prognostic Kits

The materials and reagents required for detecting the levels of expression of a p84N5 polypeptide/protein and/or mRNA in a cancer cell in a biological sample may be assembled together in a kit.

One set of kits are designed to detect the levels of expression of a p84N5 nucleic acid. Such kits of the invention will generally comprise one or more preselected primers or probes specific for p84N5. Preferably, the kits will comprise, in suitable container means, one or more nucleic acid probes or primers and means for detecting nucleic acids. In certain embodiments, such as in kits for use in Northern blotting, the means for detecting the nucleic acids may be a label, such as a radiolabel, that is linked to a nucleic acid probe itself.

Preferred kits are those suitable for use in PCR™. In PCR™ kits, two primers will preferably be provided that have sequences from, and that hybridize to, spatially distinct regions of the p84N5 gene. Preferred pairs of primers for amplifying nucleic acids are selected to amplify the sequences specified herein. Also included in PCR™ kits may be enzymes suitable for amplifying nucleic acids, including various polymerases (RT, Taq, etc.), deoxynucleotides and buffers to provide the necessary reaction mixture for amplification.

The molecular biological detection kits of the present invention, as disclosed herein, also may contain one or more of a variety of other cancer marker gene sequences as described above. By way of example only, one may mention prostate specific antigen (PSA) sequences, probes and primers.

In each case, the kits will preferably comprise distinct containers for each individual reagent and enzyme, as well as for each cancer probe or primer pair. Each biological agent will generally be suitable aliquoted in their respective containers.

The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

In further embodiments, the invention provides immunological kits for use in detecting the levels of expression of p84N5 in biological samples, e.g., in cancer cells. Such kits will generally comprise one or more antibodies that have immunospecificity for p84N5 proteins or peptides.

As the anti-p84N5 antibodies may be employed to detect p84N5 proteins or peptides and their levels, both of such components may be provided in the kit. The immunodetection kits will thus comprise, in suitable container means, a p84N5 protein or peptide, or a first antibody that binds to such a protein or peptide, and an immunodetection reagent.

In more preferred embodiments, it is contemplated that the antibodies will be those that bind to the p84N5 epitopes. MAbs are readily prepared and will often be preferred. Where proteins or peptides are provided, it is generally preferred that they be highly purified.

In certain embodiments, the p84N5 protein or peptide, or the first antibody that binds to the p84N5 protein or peptide may be bound to a solid support, such as a column matrix or well of a microtitre plate.

The immunodetection reagents of the kit may take any one of a variety of forms, including those detectable labels that are associated with, or linked to, the given antibody or antigen itself. Detectable labels that are associated with or attached to a secondary binding ligand are also contemplated. Exemplary secondary ligands are those secondary antibodies that have binding affinity for the first antibody or antigen.

Further suitable immunodetection reagents for use in the present kits include the two-component reagent that comprises a secondary antibody that has binding affinity for the first antibody or antigen (generally anti-p84N5 or p84N5), along with a third antibody that has binding affinity for the second antibody, wherein the third antibody is linked to a detectable label.

As noted above in the discussion of antibody conjugates, a number of exemplary labels are known in the art and all such labels may be employed in connection with the present invention. Radiolabels, nuclear magnetic spin-resonance isotopes, fluorescent labels and enzyme tags capable of generating a colored product upon contact with an appropriate substrate are suitable examples.

The kits may contain antibody-label conjugates either in fully conjugated form, in the form of intermediates, or as separate moieties to be conjugated by the user of the kit.

The kits may further comprise a suitably aliquoted composition of a p84N5 antigen whether labeled or unlabeled, as may be used to prepare a standard curve for a detection assay.

The kits of the invention, regardless of type, will generally comprise one or more containers into which the biological agents are placed and, preferably, suitable aliquoted. The components of the kits may be packaged either in aqueous media or in lyophilized form.

The immunodetection kits of the invention, may additionally contain one or more of a variety of other cancer marker antibodies or antigens, if so desired. Such kits could thus provide a panel of cancer markers, as may be better used in testing a variety of patients. By way of example, such additional markers could include, other tumor markers such as PSA, SeLe^{x}, HCG, as well as p53, cyclin D1, p16, tyrosinase, MAGE, BAGE, PAGE, MUC18, CEA, p27 and βPHCG.

The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the antibody, antigen, and any other reagent containers in close confinement for commercial sale. Such containers may include injection or blow-molded plastic containers into which the desired vials are retained.

### XVI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### N5 Gene Transfer Inhibits Tumor Growth and Metastasis of Human Carcinoma in Nude Mice

Materials and Methods

Cells and Culture Conditions. The FG (fast growing) human pancreatic adenocarcinoma cell line was established from COLO 357 human pancreatic carcinoma cells and is metastatic in nude mice (Vezeridis et al., 1990). Rb protein is expressed normally in COLO 357 (Barton et al., 1995). Although the status of p16Ink4 in COLO 357 has not been described, nearly all pancreatic adenocarcinomas that have been tested lack normal p16 expression (Schutte et al., 1997). The SK-OV3 (Rb positive, p16 null) (Todd et al., 2000) ovarian carcinoma cell line (SKOV3-ip1), the SAOS-2 (Rb negative) (Shew et al., 1990) and U2OS (Rb positive, low p16) (Rogatsky et al., 1997) osteosarcoma cell lines, the MDA-MB-453 (Rb positive (Kulp et al., 1996), cyclin D1 amplification (Lebwohl et al., 1994) breast carcinoma cell line, the WI38 normal human fibroblast cell line, and the 293 cell line were obtained from American Type Culture Collection. All of the cell lines express p84N5 detectable by western blot analysis, although the relative level of expression varies by as much as five fold from high expressing lines (U2OS) to low expressing lines (WI38). All cells were maintained in Dulbecco's modified Eagle's medium with 10% fetal bovine sera and antibiotics (100 unit/ml penicillin, 100 ug/ml streptomycin), in a 5% CO2 incubator at 37° C.

Animal Models. Male athymic BALB/c nude mice were purchased from the Animal Production Area of the National Cancer Institute, Frederick Cancer Research Facility (Frederick, MD). The mice were housed in a laminar flow cabinets under specific pathogen-free conditions. Animals were maintained in facilities approved by the American Association for Accreditation of laboratory Animal Care in accordance with current regulations and standards of the United States Department of Agriculture, Department of Health and Human Services and NIH.

For the subcutaneous mouse model, exponentially growing SKOV3-ip1 or FG cells were harvested after a brief exposure to trypsin then washed and resuspended in phosphate buffered saline (PBS). A total of 5×10⁵ SKOV3-ip1 or Colo357.X cells were injected subcutaneously into nude mice. Once tumor volumes reached 40-50 mm, 100 μl of adenovirus suspension (1.0×10⁸ pfu) was injected intratumorally weekly for 3 wks. PBS was used as a vehicle for sham control. Tumor diameters in three dimensions were measured weekly using a caliper and estimates of tumor volume were calculated from these measurements.

For orthotopic tumor growth, nude mice were anesthetized with methoxyflurane and placed in the supine position. An upper midline abdominal incision was made, and the pancreas was exteriorized. A total of 1×10⁶ FG cells prepared as above in 50 μl Hepes buffered saline solution (HBSS) were injected into the tail of the pancreas. Two weeks later, 50 μl of adenovirus suspension (1.0×10⁸ pfu) was injected intratumorally weekly for 2 wks. Mice were sacrificed on day 60 or when moribund. Tumors were then collected, weighed, and liver metastases counted using a dissecting microscope.

Preparation of recombinant adenovirus. The construction of the E1-deleted, replication defective adenovirus designed to express p84N5 (AdN5) or the green fluorescent protein (AdGFP) are described previously (co-pending U.S. Patent Application Serial No. 60/151,687; Yin et al., 2000). Large-scale viral preps were harvested from 293 cells at 36-40 hrs postinfection by resuspension in PBS and lysis by freeze-thaw. Virus was purified from cleared lysate by CsCl equilibrium density gradient centrifugation, and viral particles were quantitated by spectrophotometry as described previously (Huyghe et al., 1995). The recombinant adenovirus engineered to express p53 was constructed similarly (Zhang et al., 1993) and purified viral stocks were purchased from the M. D. Anderson Vector Core Laboratory. Infectious titer was estimated by an end point plaque assay on 293 cells. The concentrated virus was dialyzed against PBS plus 10% glycerol, aliquoted, and stored at −80° C.

Analysis of infected tumor cells. To measure the effect of viral infection on cell growth, cells were plated at a density of 20,000 cells/well in 12-well plates in triplicate. Cells were infected with AdN5, AdGFP or PBS as above. The following day, media was changed to remove the viral supernatant. Cells were harvested at indicated time intervals and viable cells counted using trypan blue and a hemacytometer. In vitro infected cells were analyzed for DNA fragmentation and protein expression as described (co-pending U.S. Patent Application Serial No. 60/151,687; Yin et al., 2000). Subcutaneous tumors were resected and sectioned for immunohistochemical analysis of p84N5 expression and apoptosis by terminal deoxytransferase labeling of fragmented DNA as described (Shi et al., 2000). For both western blotting and immunohistochemical analysis, the p84N5 (Durfee et al., 1994) antibody was used at a dilution of 1:1000.

Results

Sensitivity to Adenoviral-Mediated Gene Transfer. Five human tumor cell lines and one normal human cell line were used to study the effects of AdN5 infection in vitro and in vivo. The five human tumor cell lines were derived from ovarian carcinoma (SKOV3-IP1), osteosarcoma (SAOS-2 and U2OS), breast carcinoma (MDA-MB-453), and pancreatic adenocarcinoma (FG, a fast growing variant of COLO 357). The WI38 human diploid fibroblast cell line was used to model normal human cells. The sensitivity of these cell lines to adenoviral mediated gene transduction was compared by infecting them at a known multiplicity of infection (MOI) with AdGFP and measuring the percentage of cells fluorescing green the following day. At an MOI of 10, SAOS-2, USOS, MDA-MB-453, and WI38 cells exhibited greater than 90% green fluorescent cells. An MOI of 10 was used for all subsequent in vitro experiments with these lines. SKOV3-IP1 and FG cells required an MOI of 50 to exhibit greater than 90% green fluorescent cells. An MOI of 50 was used for all subsequent in vitro experiments involving these two cell lines.

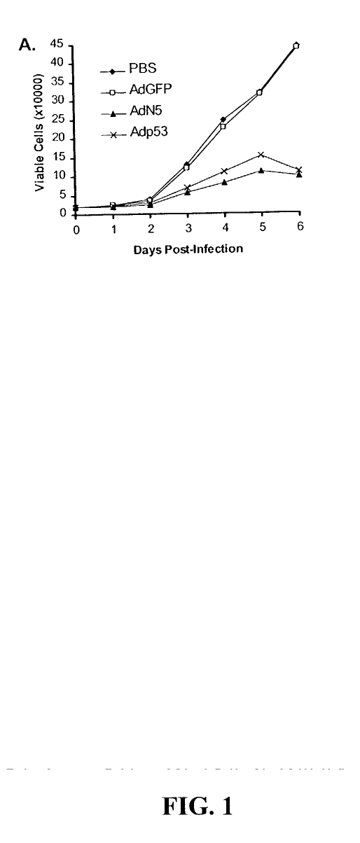

N5 Gene Transfer Inhibits Cell Proliferation In Vitro by Induction of Apoptosis. To determine the effect of AdN5 infection on the proliferation of FG cells in vitro, growth curves were measured after treatment with AdN5, Adp53, AdGFP, or PBS. Infection with AdN5 or p53 inhibited cell proliferation relative to treatment with AdGFP or PBS (FIG. 1). Significantly fewer viable cells accumulated in AdN5 or Adp53 infected cultures as early as day 3 post-infection and cell numbers declined after day 5. Inhibition of tumor cell growth by AdN5 was at least as great as with Adp53. Treatment of cells with AdGFP had no detectable affect on cell proliferation relative to PBS treated cells.

To ensure that the decrease in proliferation of AdN5 infected cells was due to an increase in N5 gene expression, infected cultures of FG cells were analyzed for p84N5 expression by western blotting. For this, the level of p84N5 expression after 12, 24 or 40 hours after treatment with either AdGFP, AdN5, or PBS was determined by western blotting. Identical blots were also probed with anti β-actin antibody to control for protein loading. An increase in p84N5 expression was observed as early as 12 hrs after infection in AdN5 infected cells relative to AdGFP infected cells. Levels of p84N5 increased 2 to 3-fold upon AdN5 infection and the increase persisted until at least 40 hrs post-infection.

Infected cells were analyzed for the presence of regularly fragmented DNA, a hallmark of apoptotic cells. For this, FG human pancreatic adenocarcinoma cells were treated with PBS or the indicated virus at an MOI of 50. Cells were collected and analyzed for the presence of fragmented DNA by agarose gel electrophoresis and ethidium bromide staining. Regularly fragmented DNA was detected in AdN5 infected cells, as indicated by the appearance of DNA ladders, which is characteristic of apoptotic cells, but not in AdGFP infected or PBS treated cells demonstrating that apoptosis is induced subsequent to AdN5 infection.

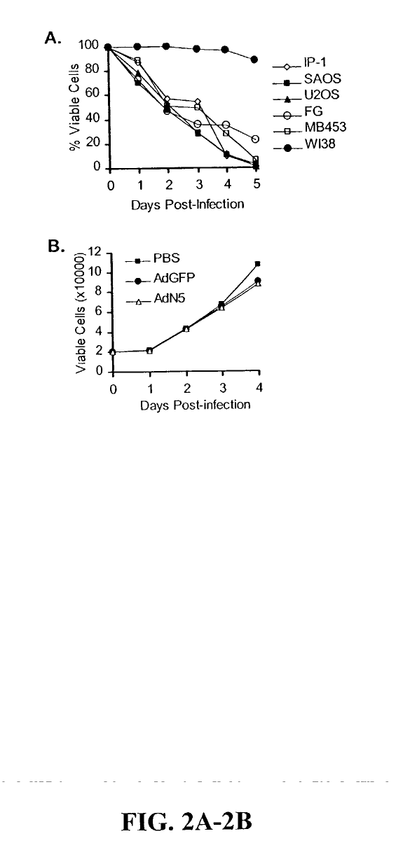

Tumor Cells are More Sensitive to the Effects of AdN5 Than Normal Cells. As Rb was shown to inhibit N5-induced apoptosis, the possibility that normal cells may be more resistant to the effects of N5 expression than tumor cells with deregulated Rb was tested. The percentage of viable cells in cultures from each of five different tumor cell lines infected with AdN5 declined from greater than 90% at the time of infection to 20% or less five days later (FIG. 2A). Although, viable cells were still detected on day 5 post-infection, less than 5% of cells in infected cultures survived and proliferated. Hence the rate of cell death in individual cells is variable. All of the tumor cell lines tested here are deregulated for the Rb growth control pathway either by Rb mutation (SAOS-2), loss of p16 expression (U2OS, FG, SK-OV3), or increased cyclin Dl expression (MDA-MB-453).

In contrast to the tumor cell lines tested, the normal human fibroblast cell line WI38 with intact Rb mediated growth control is resistant to AdN5. The percentage of viable WI38 cells infected with AdN5 was 90% or greater throughout the course of the experiment. Not only were WI38 cells less sensitive to AdN5-induced cell death, but they were also resistant to N5-induced cell cycle arrest since the growth rate of AdN5 and AdGFP infected cells was similar (FIG. 2B). The difference in AdN5 sensitivity between the tumor and normal cell lines was not due to differences in the efficiency of adenoviral mediated gene transfer since the MOI used to infect each cell line was adjusted to ensure that greater than 90% of the cells were infected.

To address the possibility that differences in sensitivity were due to differences in the levels of p84N5 expressed upon successful infection, p84N5 levels were compared two days after infection in sensitive FG tumor cells and insensitive WI38 normal cells. Both endogenous and induced levels of p84N5 were higher in the FG cell line compared to WI38. For this, FG or WI38 cells were infected with the AdGFP or AdN5 and total cell protein extracted 36 hours later. Proteins were analyzed by western blotting using a monoclonal antibody specific for p84N5 or β-actin. Densitometric comparison of the AdGFP and AdN5 infected samples indicated that the relative increase in p84N5 levels upon AdN5 infection was-similar for each cell line. In FG cells, AdN5 infection led to a 2.9 fold increase in p84N5 signal while a 3.1 fold increase was observed in WI38 cells.

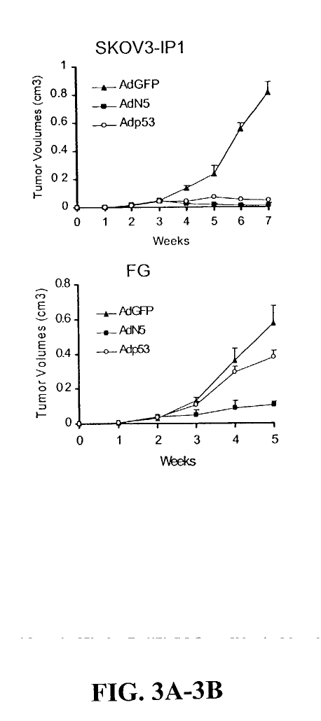

N5 Gene Transfer Inhibits Ectopic Growth of Human Ovarian and Pancreatic Cancer in Mice. The utility of N5 gene therapy in the treatment of pancreatic or ovarian cancer was tested by analyzing the effects of AdN5 infection in a subcutaneous mouse models using FG or SKOV3-ip1 cells. Viable cells were subcutaneously injected into the backs of nude mice and tumors allowed to grow for 1-2 wks until they reached a palpable size. Tumors were then treated by weekly intratumoral injection of AdN5, Adp53, or AdGFP for 3 wks. Tumor volume was monitored weekly for five weeks. The size of both FG and SKOV3-ip1 tumors was significantly reduced upon treatment with AdN5 relative to those tumors treated with AdGFP (FIG. 3A and FIG. 3B). For SKOV3-ip1 tumors, both Adp53 and AdN5 prevented significant tumor growth. Although both Adp53 and AdN5 slowed the growth of FG tumors by a stastically significant margin the AdN5 treated tumors were four-fold smaller than the Adp53 treated tumors by the end of the 5 week study.

To ensure that the reduction in tumor growth upon AdN5 treatment was due to induction of apoptosis upon successful N5 gene transfer, FG tumors were harvested and tumor sections analyzed for expression of p84N5 by immunohistochemistry and for apotosis (fragmented DNA) by TUNEL. For this, subcutaneous FG tumors were harvested two days following intratumoral injection of AdGFP or AdN5. Sections were analyzed for p84N5 expression by immunohistocytochemistry or for fragmented DNA by TUNEL. Representative sections were photographed using either a 10×or 40×objective, for immunohistocytochemistry or a 40×objective for TUNEL staining. AdN5 infected tumor cells showed an increase in nuclear p84N5 expression (immunohistocytochmistry) or positive TUNEL staining relative to AdGFP infected cells. The results are representative of numerous sections isolated from multiple tumors treated with each virus.

In contrast to AdGFP treated tumors, that express low levels of endogenous p84N5, a significant fraction of AdN5 infected tumor cells exhibited darkly stained nuclei with the anti-N5 antibody, consistent with increased p84N5 expression and successful gene transfer. In addition, tumors treated with AdN5 contained apoptotic cells, as determined by TUNEL stained nuclei and apoptotic cellular morphology, while AdGFP treated tumors did not.

N5 Gene Transfer Inhibits Orthotopic Growth and Metastasis of Human Pancreatic Cancer in Mice. To assess the effects of N5 gene therapy on the growth and metastasis of primary pancreatic tumors under more physiological conditions, the inventors tested the effects of AdN5 treatment in an orthotopic model of pancreatic adenocarcinoma (Shi and Xie, 2000; Shi et al., 1999). Beginning two weeks after implantation of FG cells into the pancreas of nude mice, tumors were treated with AdN5, AdGFP, or PBS. The mice were sacrificed on day 60 post-implantation, or when moribund, and the mass of the primary tumors was measured. AdN5 treatment reduced the mass of the resulting tumors by at least two-fold relative to PBS or AdGFP treated tumors (Table 5). This difference was statistically significant with a p value of less than 0.01 by Student's t-test. In addition, the incidence and number of liver metastasis in these mice were measured. AdN5 reduced both the incidence and mean number of liver metastasis by a statistically significant margin.

TABLE 5
N5 Gene Therapy Reduces Primary Tumor Size and the Incidence of Liver
Metastasis in an Orthotopic Mouse Model of Human Pancreatic Cancer

| | | | Liver metastasis | |
|---|---|---|---|---|
| | Primary Tumor | | | Median # |
| Treatment | Incidence | Weight (g) | Incidence | (range) |
| PBS | 10/10 | 1.44 ± 0.31 | 7/10 | 7 (0-23) |
| AdGFP | 10/10 | 1.26 ± 0.23 | 6/10 | 9 (0-15) |
| AdN5 | 10/10 | 0.62 ± 0.20^{a} | 3/10 | 0 (0-5)^{b} |

^{a}p < 0.01, student t test;
^{b}p < 0.01, Kruskal-Willis U test

### Example 2

### Combination Therapy Using N5 and p53

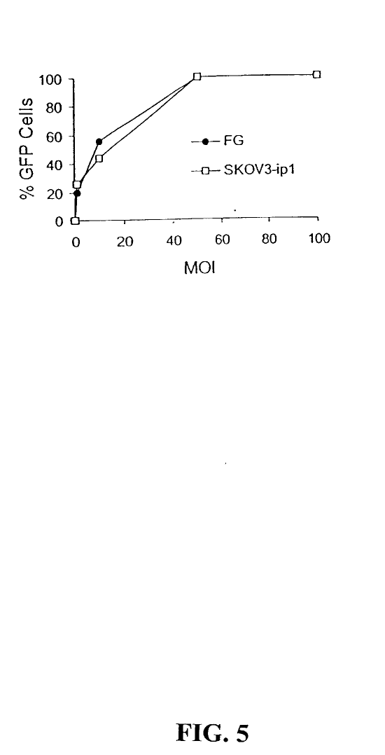

The inventors have found that treatment with AdN5 is at least as effective as treatment with adenoviral p53 (Adp53) in inhibiting tumor cell growth in vitro and in vivo for each of the cell lines tested. However, the relative sensitivity of tumor cells to N5 or p53 gene therapy varies. For example, although both the SKOV3-ip1 cells and the FG cells are equally sensitive to adenoviral mediated gene transfer, FG cells are more sensitive to AdN5 than to Adp53 (FIG. 5). On the other hand, both AdN5 and Adp53 inhibit the growth of SKOV3-ip1 tumors to a similar degree (see FIGS. 3A and 3B).

In the case of the pancreatic tumor cells, (i.e., FG cells), there is a large difference in tumor growth between AdN5 and Adp53 treated FG cells in vivo and a corresponding statistically significant difference in growth rate of similarly treated cells in vitro. Furthermore, the use of AdN5 and Adp53 together at the same total virus dose inhibits tumor growth better than either virus alone. Thus, in some embodiments, the invention contemplates combined gene therapy using both AdN5 and Adp53 or for that matter N5 gene products and p53 gene products in any viral vector or non-viral vector or method known in the art as suitable for gene-therapy to provide therapeutic methods for inhibition of tumor growth.

### Example 3

### Enhancement of Radiation Sensitivity

The inventors have also shown herein that the expression levels of p84N5 polypeptides/proteins correlate to the radiation sensitivity in tumor/cancer cells. In these studies cancer cell lines, represented by SAOS-2 (a human osteogenic sarcoma line), U2OS (a osteosarcoma cancer line), DU145 (a prostate cancer line), C33A (a cervical cancer line), and 293, were analyzed.

For the analysis, equal masses of total cell proteins were extracted from each cell line and analyzed for p84N5 expression by western blotting (see FIG. 4A). N5 was found to be expressed in each cell line examined, however the levels of expression varied considerably. For example, C33A, 293 and U2OS cells have relatively high expression levels. On the other hand, DU145 cells have barely detectable levels of p84N5 and SAOS-2 cells have moderate levels of expression. The relative sensitivity of these cells to ionization radiation were tested by clonogenecity assays.

Radiation clonogenic survival curves were plotted and compared for each cell line (see FIG. 4B). It was found that lower levels of expression of p84N5 correlate to lower radiation sensitivity.

The inventors contemplate experiments with antisense N5 cDNA to artificially reduce the levels of N5 gene products. The inventors also contemplate similar experiments with chemotherapeutic agents to determine chemotherapeutic sensitivity in relation to expression levels of p84N5.

### Example 4

### Nuclear Localization is Required for Induction of Apoptotic Cell Death by the Rb-Associated p84N5 Death Domain Protein

The mechanisms utilized to transduce apoptotic signals that originate from within the nucleus, in response to DNA damage, are not well understood. Identifying these mechanisms is important for predicting how tumor cells will respond to genotoxic radiation or chemotherapy. The Rb tumor suppressor protein is known to inhibit apoptosis triggered by DNA damage, but how it does so is unclear. As demonstrated herein above, the p84N5 death domain associates with an amino-terminal domain of Rb protein. The p84N5 death domain is required for its ability to trigger apoptotic cell death. Association with Rb protein inhibits p84N5-induced apoptosis indicating that it may be a mediator of Rb's effects on apoptosis. However, unlike other death domain-containing apoptotic signaling proteins p84N5 is localized predominantly within the nucleus of interphase cells. In the present example, the inventors tested if p84N5 requires nuclear localization in order to trigger apoptosis. To analyze the role of p84N5 in transducing apoptotic signals generated from within the nucleus, firstly the p84N5 nuclear localization signal was identified. The inventors then determined whether nuclear localization is required for p84N5-induced cell death.

Methods

A. Mapping of Nuclear Localization Signal

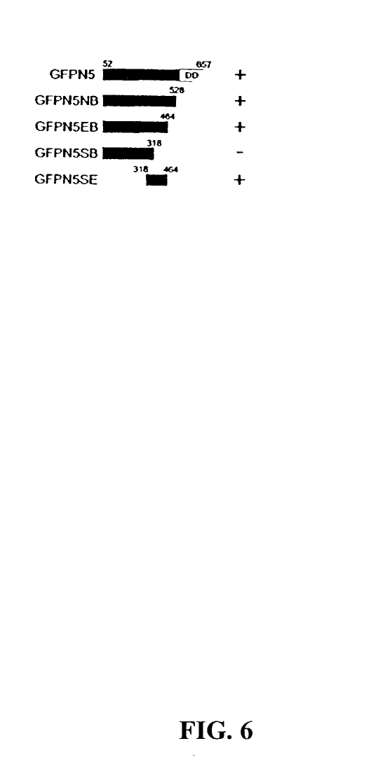

Deletion Mutants. For mapping the amino acids required for exclusive nuclear localization of p84N5, an 1800 bp fragment encoding amino acids 54 to 656 of the p84N5cDNA was subcloned in frame into the EGFP-C1 mammalian expression vector to create GFPN5. Subsequent deletion mutants were derived from this construct as follows: GFPN5NB was created by deleting the NheI-BamHI fragment of GFPN5; GFPN5EB was created by deleting the EcoRI-BamHI fragment of GFPN5; GFPN5SB was created by deleting the SalI-BamHI fragment of GFPN5; GFPN5SE is an inframe internal deletion created from GFPN5EB by removing the SalI-EcoRI fragment and inserting a SpeI linker. GFP is expressed from the empty EGFP-C1 plasmid. The schematic in FIG. 6 indicates the p84N5 amino acids included within each plasmid, the position of the p84N5 death domain, and whether the expressed fusion protein is localized predominantly within the nucleus.

Transfection and Expression. SAOS-2 cells were transfected with the plasmid DNA, including GFP; GFPN5; GFPN5NB; GFPN5EB; GFPN5SB; or GFPN5SE, using the calcium phosphate precipitation method (Wigler et al., 1979). Cells were plated on etched cover-slips one day prior to transfection. One day post-transfection, coverslips were washed twice in phosphate-buffered saline (PBS) and fixed in a solution containing 1% paraformaldehyde in PBS. Fixed cells were washed twice with PBS and once with distilled water before mounting on slides. Images were captured under fluorescent microscopy with a Hamamatsu 16-bit digital camera mounted on a Zeiss Axioplan microscope using a 636 objective.

Protein Expression Analysis. The 293 cell line was transfected as above and 2 days later resuspended in an ice-cold buffer containing 50 mM Tris, pH 7.4, 250 mM NaCl, 5 mM EDTA, 0.1% NP-40, 50 mM Naf,1 mM PMSF, 1 mg/ml leupeptin. Protein was extracted by three rounds of freeze/thaw and cell debris was pelleted by microcentrifugation. Total protein concentration of the soluble extract was determined by Bradford assay according to the manufacturer's instructions (BioRad, Hercules, Calif., USA). Twenty mg of total soluble protein for each sample was resolved by SDS±PAGE on a 10% gel. The proteins were transferred to nitrocellulose and stained for the fusion proteins using an anti-GFP antibody (Clontech, Palo Alto, Calif., USA) or β-actin as previously described (Doostzadeh-Cizeron et al., 2000a). Primary antibody was detected using a peroxidase-conjugated secondary antibody and Enhanced Chemiluminescence as described by the manufacturer (Amersham Pharmacia Biotech, Uppsala, Sweden) and compared with molecular weight standards.

B. Identification of the p84N5 Nuclear Localization Signal.

Microscopy. Transfections and fluorescent microscopy were performed as described above and photomicrographs of representative cells transfected with the plasmids were obtained. Multiple cells for each transfection examined.

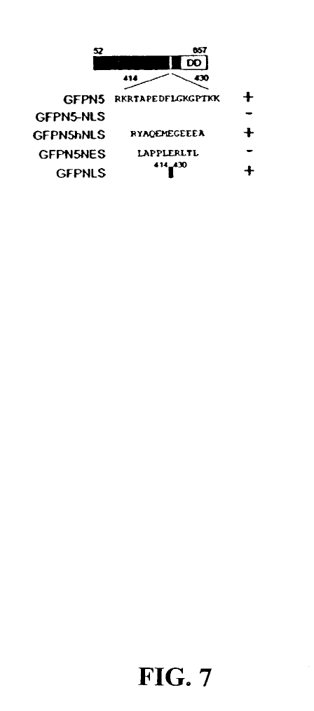

Construction of Mutant Plasmids. The mutant plasmids were constructed by PCR-mutagenesis as previously described (Fisher and Pei, 1997). The template for mutagenesis was GFPN5. GFPN5-NLS was created using the following pair of adjacent phosphorylated primers (5′-AATTATTCTCGTAGGTTTGGTA-TCTGATG-3′ (SEQ ID NO. 6) and 5′-ATTCTGACGGGAAATGAGGAGTTA-ACAAGG-3′ (SEQ ID NO. 7)). GFPN5+NLS was created using the following pair of adjacent phosphorylated primers (5′-CATCTCCTGGGCAT-AACGAATTATTCTCGTAGGTTTGGTATC-3′ (SEQ ID NO. 8) and 5′-GAA-GGCGAAGAAGAAGCCATTCTGACGGGAAATGAGGAGTTA-3′ (SEQ ID NO. 9)). GFPN5+NES was created using the following pair of adjacent phosphorylated primers (5′-GAACTTCTTCGTCATAATTATTCTCGTAGGTTTGGTATC-3′ (SEQ ID NO. 10) and 5′-GGCACGCTCACGATCATTCTGACGGGAAATGAGGAG-3′ (SEQ ID NO. 11)). DNA sequencing was used to confirm the mutagenesis. The schematic in FIG. 7 indicates the amino acid sequence of the putative bipartite p84N5 NLS, the amino acids contained in the mutant derivatives, and whether they are localized predominantly within the nucleus.

Western Blotting. Total cell protein extracted from 293 cells transfected with each of the indicated plasmid constructs was analysed by Western blotting as described above.

Results

Mapping of the Nuclear Localization Signal. To facilitate identifying the p84N5 nuclear localization signal (NLS), the near full-length N5 cDNA was sub-cloned into EGFP-C1 to create a plasmid designed to express a GFPN5 fusion protein. This fluorescent protein contains GFP-fused to the amino-terminus of p84N5 amino acids 52to 657 (FIG. 6). Following transfection of GFPN5 into SAOS-2 cells, 10±15% of the cells exhibited a fluorescent signal and this signal was localized exclusively within the nucleus. In contrast, cells transfected with EGFP-C1 exhibit fluorescence throughout the cell. Although the GFPN5 fusion protein is correctly localized to the nucleus, it does not exhibit the punctate staining pattern typically observed upon immuno fluorescent staining of endogenous p84N5 (Durfee et al., 1994). This is likely due to the fact that the GFPN5 protein accumulates to higher levels than the endogenous protein. A small fraction of GFPN5 positive cells, less than 5%, show green fluorescence outside the nucleus. Based on their morphology, these cells appeared to be undergoing apoptosis. Localization of endogenous p84N5 has been observed to change during apoptosis (Doostzadeh-Cizeron et al., 1999).

A series of deletions within the N5 coding region of GFPN5 were created, expressed, and analysed for nuclear localization by microscopy as described above (FIG. 6). A deletion mutant lacking p84N5 amino acids 318 to 657 (GFPN5SB) expressed a fluorescent signal throughout the cell while a mutant lacking amino acids 464 to 657 (GFPN5EB) expressed a protein localized within the nucleus. Hence, the p84N5 NLS is located within amino acids 318 to 464. The GFPN5SE mutant that expresses a nuclear localized protein containing only amino acids 318 to 464 confirmed this. Western blot analysis of protein extracts from similarly transfected cells using an anti-GFP antibody indicated that each plasmid expressed a protein of the expected apparent molecular mass. Amino acids 318 to 464 of p84N5 contain a potential bipartite NLS with the sequence RKRTA-PEDFLGKGPTKK (SEQ ID NO. 12) that spans amino acids 414 to 430 (FIG. 7).

Identification of the Nuclear Localization Signal. To test whether this sequence is necessary and sufficient for localizing GFPN5 to the nucleus, an additional series of mutants were constructed containing alterations within this sequence. GFPN5-NLS is an in-frame deletion of sequences encoding amino acids 414 to 430. GFPN5NES replaces these sequences with amino acids that are similar to the nuclear export signal (NES) of HIV-1 Rev (Fischer et al., 1995). Both of these mutants expressed a fluorescent protein that failed to localize within the nucleus. The distribution of GFP signal from these mutants between the nucleus and the cytoplasm varied from cell to cell. In some cells there appeared to be little nuclear signal. In other cells, however, the signal appeared to be uniformly distributed throughout the cells similar to GFP. Although GFPN5NES contained an NES, it failed to exclude GFP signal from the nucleus to any greater extent than the GFPN5-NLS mutant. Replacing p84N5 amino acids 414 to 430 withan unrelated NLS (RYAQEMEGEEEA (SEQ ID NO. 13)) restored nuclear localization of the fusion protein expressed from the GFPN5hNLS mutant. A GFP fusion protein expressed from the GFPNLS mutant containing just N5 amino acids 414 to 430 was also localized exclusively to the nucleus. The fusion proteins expressed by all of the mutant plasmids were analysed by Western blotting of total protein extracted from similarly transfected cells. All of the plasmids expressed a fusion protein of predicted molecular mass confirming that the correct protein was made in each case. Thus, amino acids 414 to 430 contain the sequence that is both necessary and sufficient to localize p84N5 to the nucleus.

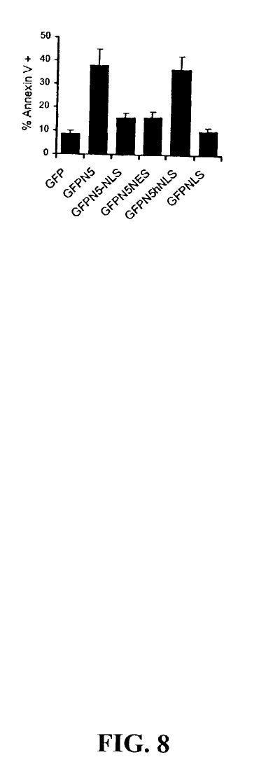

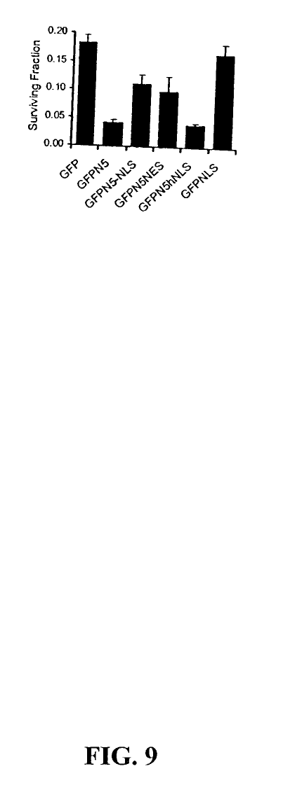

Induction of Apoptosis. Expression of p84N5 in C33A cells induces apoptotic cell death that can be detected by annexinV staining (Doostzadeh-Cizeron et al., 2000a). To test the function of GFPN5 and the mutant derivatives, their ability to trigger apoptosis upon expression in C33A cells was analyzed. Annexin V staining was detected in about 40% of cells successfully transfected with GFPN5 compared to less than 10% of cells expressing the negative control proteins GFP or GFPNLS (FIG. 8). Both mutant fusion proteins that fail to localize within the nucleus, GFPN5-NLS and GFPN5NES, had reduced ability to trigger apoptosis as indicated by annexin V staining on only about 15% of successfully transfected cells. Restoration of nuclear localization with the unrelated NLS in the GFPN5hNLS fusion protein also restored its ability to trigger annexin V staining in a percentage of cells comparable to that observed with wild type GFPN5. Each of the GFPN5 fusion proteins accumulates to approximately the same level. The differences in function observed for the mutant fusion proteins, therefore, are not due to differences in protein level.

Recluction of Clonogenic Potential. Since expression of p84N5 can induce a G2/M cell cycle arrest (Doostzadeh-Cizeron et al., 2001) as well as apoptosis, p84N5 can reduce the proliferative capacity of cells (Doostzadeh-Cizeron et al., 1999) by multiple mechanisms. To exclude the possibility that the failure of the GFPN5-NLS or GFPN5NES proteins to trigger apoptosis was merely due to a delay in the kinetics-of apoptosis, their ability to reduce the clonogenic potential of cells was tested. Cells expressing GFPN5 had at least a five-fold decrease in their ability to form proliferating colonies compared to cells expressing GFP (FIG. 9). Cells expressing either the GFPN5-NLS or GFPN5NES fusion proteins were able to form proliferating colonies with 3±4 -fold greater efficiency than cells expressing GFPN5. Restoration of nuclear localization in the GFPN5hNLS mutant restores the ability of the fusion protein to reduce the clonogenic potential of expressing cells to levels similar to that observed with wild type GFPN5. Cells expressing mutant proteins that did not localize to the nucleus, GFPN5-NLS and GFPN5NES, have a lower percentage of apoptotic cells and greater clonogenic potential than cells expressing wild type GFPN5. Although cells expressing GFPN5-NLS or GFPN5NES are both compromised in their function, they do demonstrate activity in these assays above the negative controls GFP and GFPNLS. This is likely due to the fact that some protein makes it into the nucleus in some transfected cells. The relative level of nuclear fluorescence observed in cells expressing GFPN5-NLS and GFPN5NES proteins varies from cell to cell. Some cells appear to have a uniform distribution of protein throughout the cell, including the nucleus. Other cells appear to have very little protein within the nucleus (similar to GFPN5-NLS or GFPN5NES). Although these mutant proteins are not localized predominantly within the nucleus, neither are they excluded from the nucleus. There is no consistent difference in the relative amount of nuclear fluorescence observed between any of the mutant proteins or GFP even though GFPN5NES contains an NES. Hence, some cells expressing GFPN5-NLS or GFPN5NES may accumulate sufficient nuclear protein to induce cell cycle arrest and apoptosis in the absence of an NLS. This may explain their reduced, but detectable, level of activity in these assays. Thus, the ability of GFPN5 to trigger apoptotic cell death and inhibit clonogenicity correlates with its nuclear localization. Since amino acids 414 to 430 are not required for p84N5 function, as indicated by the GFPN5hNLS mutant, loss of function in the GFPN5-NLS protein is due to lack of nuclear localization rather than to disruption of a required functional domain. The GFPNLS fusion protein containing only the N5 NLS is localized to the nucleus but fails to function like GFPN5. This finding demonstrates that nuclear localization of GFP alone is insufficient for induction of apoptosis or inhibition of clonogenicity. N5 protein can mediate apoptotic cell death and this ability requires an intact death domain located in the C-terminus of the protein (Doostzadeh-Cizeron et al., 1999). In other apoptotic signaling pathways, the death domain species required protein interactions (Wolfand Green, 1999; Yuan, 1997).

The known death domain-containing proteins involved in apoptosis are typically not localized to the nucleus and function in signaling pathways originating outside the nucleus. Thus, the present invention demonstrates that p84N5 is a unique death domain-containing protein that functions within the nucleus to trigger apoptosis. Furthermore, p84N5 associates with the nuclear Rb tumor suppressor protein and this association inhibits p84N5-induced apoptosis (Doostzadeh-Cizeron et al., 1999). The p84N5 sequences required for Rb association overlap with its death domain (Durfee et al., 1994). Rb, therefore, may inhibit p84N5-induced apoptosis by precluding required death domain-mediated interactions between p84N5 and other apoptotic signaling molecules. Rb is known to inhibit apoptosis initiated from within the nucleus; for example, Rb inhibits apoptosis triggered by ionizing radiation (Haas-Kogan et al., 1995). The mechanisms used by Rb to inhibit apoptosis are not completely understood. The present inventors contemplate that p84N5 participates in an apoptotic signaling pathway initiated from within the nucleus that is regulated by Rb. Consistent with this p84N5-induced apoptosis has a pattern of caspase, NF-kB, and cell cycle checkpoint activation that is similar to that observed in response to some forms of DNA damage known to be regulated by Rb (Doostzadeh-Cizeron et al., 2000a; Doostzadeh-Cizeron et al., 2001). Further, the amino-terminal domain of Rb is not only required to bind p84N5, but is also required to regulate the cell cycle and apoptotic responses of p53 null cells to ionizing radiation. Thus, p84N5 mediates some of Rb's inhibitory effects on apoptotic responses triggered by DNA damage.

### Example 5

### Clinical Trials

This example is concerned with the development of human treatment protocols for anticancer therapy for pancreatic and ovarian cancers using the N5 gene-therapy either alone or in combination with other therapeutic agents. In some specific embodiments, the second therapeutic agent is p53 based gene or protein therapy. In other specific embodiments, the second therapeutic agent is a radiation or chemotherapeutic agent. One of skill in the art will also recognize that any other adjunct cancer therapy known is contemplated as useful in combination or conjunction with the present therapeutic methods.

The various elements of conducting a clinical trial, including patient treatment and monitoring, are known to those of skill in the art and in light of the present disclosure. The following information is being presented as a general guideline for use in establishing the therapies using the p84N5 encoding nucleic acids and/or protein products described herein alone or in combinations with other adjunct treatments used routinely in cancer therapy in clinical trials.

Candidates for the phase 1 clinical trial will be patients with pancreatic or ovarian cancers on which all conventional therapies have failed. Approximately 100 patients will be treated initially. Their age will range from 16 to 90 (median 65) years. Patients will be treated, and samples obtained, without bias to sex, race, or ethnic group. For this patient population of approximately 41% will be women, 6% will be black, 13% Hispanic, and 3% other minorities. These estimates are based on consecutive cases seen at MD Anderson Cancer Center over the last 5 years.

Optimally the patient will exhibit adequate bone marrow function (defined as peripheral absolute granulocyte count of >2,000/mm³ and platelet count of 100, 000/mm³, adequate liver function (bilirubin 1.5 mg/dl) and adequate renal function (creatinine 1.5 mg/dl).

Research samples will be obtained from peripheral blood or marrow under existing approved projects and protocols. Some of the research material will be obtained from specimens taken as part of patient care.

The N5 gene-therapy treatments described above will be administered to the patients regionally or systemically on a tentative weekly basis. A typical treatment course may comprise about six doses delivered over a 7 to 21 day period. Upon election by the clinician the regimen may be continued with six doses every three weeks or on a less frequent (monthly, bimonthly, quarterly, etc.,) basis. Of course, these are only exemplary times for treatment, and the skilled practitioner will readily recognize that many other time-courses are possible.

The modes of administration may be local administration, including, by intratumoral injection and/or by injection into tumor vasculature, intratracheal, endoscopic, subcutaneous, and/or percutaneous. The mode of administration may be systemic, including, intravenous, intra-arterial, intra-peritoneal and/or oral administration.

The p84N5 encoding nucleic acid constructs will be administered at appropriate dosages as determined by a trained physician intravenously or by other routes as discussed above. In some embodiments, the p84N5 protein or polypeptide may be administered. Of course, the skilled artisan will understand that while these dosage ranges, provide useful guidelines appropriate adjustments in the dosage depending on the needs of an individual patient factoring in disease, gender, age and other general health conditions will be made at the time of administration to a patient by a trained physician. The same is true for means of administration, routes of administration as well.

To monitor disease course and evaluate the cancer cell killing it is contemplated that the patients should be examined for appropriate tests every month. To assess the effectiveness of the drug, the physician will determine parameters to be monitored depending on the type of cancer/tumor and will involve methods to monitor reduction in tumor mass by for example computer tomography (CT) scans, detection of the induction of cell death in the tumor, and in some cases the additional detection of other tumor markers such as CA 19.9, CEA, TPA and CA 242, which are markers of panceratic cancer or CA 125 which is a marker of ovarian cancers. Tests that will be used to monitor the progress of the patients and the effectiveness of the treatments include: physical exam, X-ray, blood work, bone marrow work and other clinical laboratory methodologies. The doses given in the phase 1 study will be escalated as is done in standard phase 1 clinical phase trials, i.e. doses will be escalated until maximal tolerable ranges are reached.

Clinical responses may be defined by acceptable measure. For example, a complete response may be defined by complete disappearance of the cancer cells whereas a partial response may be defined by a 50% reduction of cancer cells or tumor burden.

The typical course of treatment will vary depending upon the individual patient and disease being treated in ways known to those of skill in the art. For example, a patient with pancreatic adenocarcinoma might be treated in four week cycles, although longer duration may be used if no adverse effects are observed with the patient, and shorter terms of treatment may result if the patient does have side effects.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Patent Serial No. 60/151,687

U.S. patent Ser. No. 09/653,465

U.S. Pat. No. 3,817,837

U.S. Pat. No. 3,850,752

U.S. Pat. No. 3,939,350

U.S. Pat. No. 3,996,345

U.S. Pat. No. 4,196,265

U.S. Pat. No. 4,215,051

U.S. Pat. No. 4,275,149

U.S. Pat. No. 4,277,437

U.S. Pat. No. 4,366,241

U.S. Pat. No. 4,578,770

U.S. Pat. No. 4,596,792

U.S. Pat. No. 4,599,230

U.S. Pat. No. 4,599,231

U.S. Pat. No. 4,601,903

U.S. Pat. No. 4,608,251

U.S. Pat. No. 4,816,567

U.S. Pat. No. 4,867,973

U.S. Pat. No. 5,565,332

U.S. Pat. No. 5,693,762

European Application No. 0273085

Abbondanzo, Ann Diagn Pathol, 3(5):318-327, 1999.

Allred et al., Arch Surg, 125(1):107-13, 1990.

Arcone, et al., Nucl. Acids Res., 16(8): 3195-3207, 1988.

Aspinall and Lemoine, Ann: Oncol., 10(14):188-192, 1999.

Baichwal and Sugden, In. Gene Transfer, Kucherlapati R, ed., New York, Plenum Press, pp. 117-148, 1986.

Bartlett et al., Proc. Nat'l Acad. Sci. USA, 93:8852-8857, 1996.

Barton et al., Mol. Carcinog., 13: 61-69, 1995.

Benvenisty and Neshif, Proc. Nat'l Acad. Sci. USA, 83:9551-9555, 1986.

Boldin et al., J. Biol. Chem. 270:387-391, 1995.

Bookstein et al., Proc. Nat'l Acad. Sci. USA. 87:7762-7766, 1990a.

Bookstein et al., Science 247,712-715, 1990b.

Bouvet et al., Annals of Surgical Oncology, 5: 681-688, 1998.

Brown et al., Immunol Ser, 53:69-82, 1990.

Bookstein and Lee, Crit Rev Oncog., 2(3):211-27, 1991.

Buchkovich et al., Cell 58, 1097-1105, 1989.

Campbell, In: Monoclonal Antibody Technology, Laboratory Techniques in Biochemistry and Molecular Biology, Burden and Von Knippenberg (Eds.), Elseview, Amsterdam, 13:71-74; 75-83, 1984.

Carter and Flotte, Ann. N.Y. Acad. Sci., 770:79-90, 1995.

Chang et al., Hepatology, 14:134A, 1991.

Chatterjee, et al., Ann. N.Y. Acad. Sci., 770:79-90, 1995.

Chen and Mixson, Front. Biosci., 3:D997-D1004, 1998.

Chen and Okayama, Mol. Cell Biol., 7:2745-2752, 1987.

Chen et al., Cell Growth Diff. 3,119-125, 1992.

Chen et al., Cell 58, 1193-1198, 1989.

Clary and Lyerly, Surg. Oncol. Clin. N. Am., 7:217-249, 1998.

Clayman, “The current status of gene therapy,” Seminars in Oncology, 27:39-43, 2000.

Cleveland and Ihle, Cell 81:479-482, 1995.

Coffin, In: Virology, ed., New York: Raven Press, pp. 1437-1500, 1990.

Coupar et al., Gene, 68:1=10, 1988.

Dani, et al., J. Biol. Chem., 264: 10119-10125, 1989.

DeCaprio et al., Cell 54, 275-283, 1988.

DeCaprio et al., Cell 58,1085-1095, 1989.

Defeo-Jones et al., Nature 352, 251-254, 1991.

Doostzadeh-Cizeron et al., Mol. Biol. Cell, 10:3251-3261, 1999.

Doostzadeh-Cizeron et al., J. Biol. Chem., 276: 1127-1132, 2001.

Doostzadeh-Cizeron et al., J. Biol. Chem., 275: 25336-25341, 2000a.

Doostzadeh-Cizeron et al., J. Biol. Chem., in press, 2000b.

Dubensky et al., Proc. Nat'l Acad. Sci. USA, 81:7529-7533, 1984.

Durfee et al., J. Cell Biol., 127: 609-622, 1994.

Dyson et al., Science 243, 934-937, 1989.

Evans et al., In: Cancer Principles and Practice of Oncology, Devita et al., (eds.), Oncology, 1054-1087, NY: Lippincot-Raven, 1054-1087. 1997.

Favrot et al., Gene Ther., 5:728-739, 1998.

Fechheimer et al., Proc. Nat'l Acad. Sci. USA, 84:8463-8467, 1987.

Feinstein et al., Trends Biochem. Sci., 20:342-344, 1995.

Ferkol et al., FASEB J., 7:1081-1091, 1993.

Ferrari et al., J. Virol., 70:3227-3234, 1996.

Fischer et al., Cell, 82:75-483, 1995.

Fisher and Pei, Biotechniques, 23:570-574.14, 1997.

Fisher et al., J. Virol., 70:520-532, 1996.

Flotte et al., Proc. Nat'l Acad. Sci. USA, 90:10613-10617, 1993.

Fraley et al., Proc. Nat'l Acad. Sci. USA, 76:3348-3352, 1979.

Friedmann, Science, 244:1275-1281, 1989.

Friend et al., Nature (London) 323, 643-646, 1986.

Fung et al., Science 236, 1657-1661, 1987.

Galanis et al., Crit. Rev. Oncol Hematol., 38(3): 177-192, 2001.

Gefter et al., Somatic Cell Genet, 3(2):231-6, 1977.

Ghosh and Bachhawat, In: Liver diseases, targeted diagnosis and therapy using specific receptors and ligands, (Wu andWu (Eds), NY: Marcel Dekker, 87-104, 1991.

Goding, In: Monoclonal Antibodies: Principles and Practice, 2d., Orl. Fla., Academic Press 60-61, 65-66, 71-74, 1986.

Goodman et al., Blood, 84:1492-1500, 1994.

Goodrich et al., Can. Res. 52, 1968-1973, 1992.

Goodrich et al., Cell 67, 293-302, 1991.

Gopal, Mol. Cell Biol., 5:1188-1190, 1985.

Gossen and Bujard, Proc. Nat'l Acad. Sci. USA, 89:5547-5551, 1992.

Gossen et al., Science, 268:1766-1769, 1995.

Graham and Van Der Eb, Virology, 52:456-467, 1973.

Haas-Kogan et al., EMBO J. 14, 461-472, 1995.

Harbour et al., Science 241; 353-357, 1988.

Harland and Weintraub, J. Cell Biol., 101: 1094-1099, 1985.

Harlow and Lane, Antibodies: A Laboratory manual, Cold Spring Harbor Lab., 1988.

Harris et al., Cancer Gene Ther., 3:121-130, 1996.

Hay et al., J. Mol. Biol., 175:493-510, 1984.

Hearing and Shenk, J. Mol. Biol. 167:809-822, 1983.

Hearing et al., J. Virol., 67:2555-2558, 1987.

Hedlund et al., Cell Death & Differ., 6: 175-182, 1999.

Heise and Kirn, J. Clin. Investig., 105: 847-851, 2000.

Herrmann et al., Exp. Cell Res., 237:101-109, 1997.

Hoon et al., J. Urol., 150(6):2013-2018, 1993.

Horowitz et al., Science 243, 937-940, 1989.

Horwich et al., Virol., 64:642-650, 1990.

Huetal., EMBO J. 9,1147-1155, 1990.

Huang et al., Nature 160-162, 1991.

Huang et al., EMBO J. 9, 1815-1822, 1990.

Huang et al., Science 242, 1563-1566, 1988.

Hunt et al., Proc. Nat'l Acad. Sci. USA, 83:3786-3790, 1986.

Huyghe et al., Hum. Gene Ther., 6: 1403-1416, 1995.

Inouye et al., Nucleic Acids Res., 13:3101-3109, 1985.

Itoh et al., J. Biol. Chem. 268:10932-10937, 1993.

Jacobson et al., Cell, 88: 347-354, 1997.

Joki et al., Human Gene Ther., 6:1507-1513, 1995.

Kaelin et al., Cell 64, 521-532 1991.

Kageyama, et al., J. Biol. Chem., 262(5):2345-2351, 1987.

Kaneda et al., Science, 243:375-378, 1989.

Kaplitt et al., Nat'l Genet., 8:148-153, 1994.

Kato et al, J. Biol. Chem., 266:3361-3364, 1991.

Kessler et al., Proc. Nat'l Acad. Sci. USA, 93:14082-14087, 1996.

Kigawa and Terakawa, Adv. Exp. Med. Biol., 465:207-214, 2000.

Kimura et al., Anticancer Res., 17: 879-883, 1997.

Kim, Oncogene, 19: 6660-6669, 2000.

Klein et al., Nature, 327:70-73, 1987.

Knudson, Proc. Nat'l Acacl. Sci., 68:820-23, 1971.

Koeberl et al., Proc. Nat'l Acad. Sci. USA, 94:1426-1431, 1997.

Kohler and Milstein, Nature, 256(5517):495-7, 1975.

Kohler and Milstein, Eur J Immunol, 6(7):511-9, 1976.

Korc, Surg. Oncol. Clin. N. Am., 7:25-41, 1998.

Korhonen et al., Blood, 86:1828-1835, 1995.

Kulp et al., Exp. Cell Res., 229: 60-68, 1996.

Le et al., Oncogene, 16:1839-1849, 1998.

Lebwohl et al., Oncogene, 9: 1925-1929.

Lee et al., Proc. Natl. Acad. Sci. U.S.A., 96:9136-9141, 1999.

Lee et al., Cold Spring Harbor Symp. Quanti. Biol.: Cell. Cycle. 61, 211-217, 1991.

Lee et al., Nature 329, 642-645, 1987b.

Lee et al., Science 241, 218-221, 1988.

Letsou et al., Proc. Nat'l Acad. Sci. USA 88:810-814, 1991.

Levrero et al., Gene, 101:195-202, 1991.

Lin et al., EMBO J. 10, 857-864, 1991.

Liu et al., Cancer Res., 54:3662-3667, 1994.

Mann et al., Cell, 33:153-159, 1983.

Matsubara et al., Cancer Research, 61, 6012-6019, 2001.

McCown et al., Brain Res., 713:99-107, 1996.

Meng et al., Clin. Cancer Res., 4:251-259, 1998.

Mizukami et al., Virology, 217:124-130, 1996.

Nakamura et al., In: Handbook of Experimental Immunology (4^{th} Ed.), Weir, Herzenberg, Blackwell, Herzenberg, (eds). Vol. 1, Chapter 27, Blackwell Scientific Publ., Oxford, 1987.

Nicolas and Rubenstein, In: Vectors: A survey of molecular cloning vectors and their uses, Rodriguez and Denhardt (eds.), Stoneham: Butterworth, pp. 493-513, 1988.

Nicolau and Sene, Biochim. Biophys. Acta, 721:185-190, 1982.

Nicolau et al., Methods Enzymol., 149:157-176, 1987.

Olivierio, et al., EMBO J., 6(7):1905-1912, 1987.

Pape and Kim, Mol. Cell. Biol., 974-982, 1989.

Paskind et al., Virology, 67:242-248, 1975.

Perales et al., Proc. Nat'l Acad. Sci. 91:4086-4090, 1994.

Ping et al., Microcirculation, 3:225-228, 1996.

Poli and Cortese, Proc. Nat'l Acad. Sci. USA, 86:8202-8206, 1989.

Potter et al., Proc. Nat'l Acad. Sci. USA, 81:7161-7165, 1984.

Prowse and Baumann, Mol Cell Biol, 8(1):42-51, 1988.

Radler et al., Science, 275:810-814, 1997.

Ravindranath and Morton, Intern. Rev. Immunol., 7: 303-329, 1991.

Remington's Pharmaceutical Sciences, 15^{th} ed., pages 1035-1038 and 1570-1580, Mack Publishing Company, Easton, Pa., 1980.

Renan, Radiother. Oncol., 19:197-218, 1990.

Ridgeway, In: Vectors: A survey of molecular cloning vectors and their uses, Rodriguez R L, Denhardt D T, ed., Stoneharn:Butterworth, pp. 467-492, 1988.

Rippe et al., Mol. Cell Biol., 10:689-695, 1990.

Rodrigez et al., Cancer Res., 57: 2559-2563, 1997

Rogatsky et al., Mol. Cell. Biol., 17: 3181-3193, 1997.

Ron et al., Mol. Cell. Biol., 2887-2895, 1991.

Rosenberg et al., Ann. Surg. 210(4):474-548, 1989.

Rosenberg et al., N. Engl. J. Med., 319:1676, 1988.

Roth et al., Oncology, 13:148-154, 1999.

Roux et al., Proc. Nat'l Acad. Sci. USA, 86:9079-9083, 1989.

Rustgi et al., Nature 352,541-544, 1991.

Samulski et al., J. Virol., 61(10):3096-3101, 1987.

Schutte et al., Cancer Res., 57: 3126-3130, 1997.

Shelton and Wasserman, Cell 72, 515-525, 1993.

Shew et al., Proc. Natl. Acad. Sci. USA, 87: 6-10, 1990.

Shi and Xie Int. J. Oncol., 17:217-225, 2000.

Shi et al., Clin. Cancer Res., 5:3711-3721, 1999.

Shi et al., Cancer Res., 60:2579-2583, 2000.

Sumegi et al., Cell Growth Differ. 1:247-250, 1990.

Swisher et al., J. Natl. Cancer, 91: 763-771, 1999.

Takahashi et al., Proc. Nat'l Acad. Sci. USA. 88, 5257-5261, 1991.

Tartaglia et al., Cell 74, 845-853, 1993.

Temin, In: Gene Transfer, Kucherlapati (ed.), New York: Plenum Press, pp. 149-188, 1986.

Tibbetts Cell, 12:243-249, 1977.

Todd, et al., Oncogene, 19: 258-264, 2000.

Toguchida et al., Cancer Res. 48,3939-943, 1988.

Tur-Kaspa et al., Mol. Cell Biol., 6:716-718, 1986.

Vaux et al., Cell, 76:777-779, 1994.

Vezeridis et al., J. Surg. Res., 48:51-55, 1990.

Vinyals et al., Gene Ther., 6:22-33, 1999.

Wagner et al., Proc. Nat'l Acad. Sci. 87(9):3410-3414, 1990.

Walther and Stein, J. Mol. Med, 74:379-392, 1996.

Wang et al., Cell Growth Diff 1, 429-437, 1990.

Wattet al., Proc. Nat'l Acad. Sci., 83(2): 3166-3170, 1986.

Whyte et al., Nature 334, 124-129, 1988.

Wiegmann et al., Cell 78:1005-1015, 1994.

Wigler et al., Cell, 16:777-785, 1979.

Wilson, et al., Mol. Cell. Biol., 6181-6191, 1990.

Wolf and Green, J. Biol. Chem. 274:20049-20052, 1999.

Wong et al., Gene, 10:87-94, 1980.

Wu and Wu, Adv. Drug Delivery Rev., 12:159-167, 1993.

Wu and Wu, Biochem., 27:887-892, 1988.

Wu and Wu, J. Biol. Chem., 262:4429-4432, 1987.

Xiao et al., J. Virol., 70:8098-8108, 1996.

Yang et al., Proc. Nat'l Acad. Sci USA, 87:9568-9572, 1990.

Yin et al., Cancer Gene Ther., 7:985-990, 2000.

Yokota et al., Oncogene 3, 471-475, 1988.

Yu et al., Cancer Res., 59: 1498-1504; 4200-4203, 1999

Yuan, Curr. Opin. Cell Biol. 9:247-251, 1997.

Zechner, et al., gMol. Cell. Biol., 2394-2401, 1988.

Zhang et al., Biotechniques, 15: 868-872, 1993.

Zimmerman, In Ophthalmic Pathology: an Atlas and Textbook, W. H. Spencer (ed.), Vol. II, Philadelphia: W. B. Saunders Co., 1292-1351, 1985.

What is claimed is:

1. A method of inducing apoptosis in a pancreatic or an ovarian cancer cell, comprising contacting the cell with a recombinant vector encoding a p84N5 death domain operably linked to a promoter that functions in the cell.

2. The method of claim 1, wherein the pancreatic cancer cell is a pre-cancerous pancreatic cell.

3. The method of claim 1, wherein the pancreatic cancer cell is a metastatic pancreatic cell.

4. The method of claim 1, wherein the pancreatic cancer cell is a malignant pancreatic cell.

5. The method of claim 4, wherein the malignant pancreatic cancer cell is a ductal adenocarcinoma cell, a mucinous cystadenocarcinoma cell, an acinar carcinoma cell, an unclassified large cell carcinoma cell, a small cell carcinoma cell, or a pancreatoblastoma cell.

6. The method of claim 4, wherein the malignant pancreatic cancer cell is an intraductal papillary neoplasm cell, a mucinous cystadnoma cell, or a papillary cystic neoplasm cell.

7. The method of claim 1, wherein the ovarian cancer cell is a pre-cancerous ovarian cell.

8. The method of claim 1, wherein the ovarian cancer cell is a metastatic ovarian cell.

9. The method of claim 1, wherein the ovarian cancer cell is a malignant ovarian cell.

10. The method of claim 1, wherein the ovarian cancer cell is an carcinoma cell, a serous cell, a mucinous cell, an endometrioid cell, a clear cell mesonephroid cell, a Brenner cell, or a mixed epithelial cell.

11. The method of claim 1, wherein the cancer cell lacks functional caspase-3.

12. The method of claim 1, wherein the cancer cell is retinoblastoma negative.

13. The method of claim 1, wherein the cancer cell is comprised in a tumor.

14. The method of claim 13, wherein the cell is comprised in an animal.

15. The method of claim 14, wherein the animal is human.

16. The method of claim 15, further defined as a method of treating cancer.

17. The method of claim 15, further defined as a method of preventing cancer.

18. The method of claim 1, wherein the p84N5 death domain comprises a p84N5 sequence up to and including full length p84N5 sequences.

19. The method of claim 1, wherein the p84N5 death domain comprises the sequence shown in SEQ ID NO:1.

20. The method of claim 1, wherein the contacting comprises administering the recombinant vector encoding the p84N5 death domain.

21. The method of claim 20, wherein the recombinant vector is a viral vector.

22. The method of claim 21, wherein the viral vector is an adenoviral vector, adeno-associated viral vector, retroviral vector, lentiviral vector, herpes viral vector, papilloma viral vector, or hepatitis B viral vector.

23. The method of claim 21, wherein the viral vector is an adenovirus.

24. The method of claim 23, wherein the adenovirus is administered at a dose of about 10¹⁰ to about 10¹² pfu.

25. The method of claim 16, further comprising treating the human with a second agent, wherein the second agent is a therapeutic polypeptide, a nucleic acid encoding a therapeutic polypeptide, a chemotherapeutic agent, an immunotherapeutic agent, or a radiotherapeutic agent.

26. The method of claim 25, wherein the therapeutic polypeptide or a nucleic acid encoding a therapeutic polypeptide is p53.

27. The method of claim 25, wherein the second agent is administered simultaneously with the recombinant vector encoding p84N5.

28. The method of claim 25, wherein the second agent is administered at a different time than the recombinant vector encoding p84N5.

29. The method of claim 15, wherein the contacting is by intravenous, intraartetial, intraperitoneal, intradermal, intratumoral, intramuscular, oral, dermal, nasal, buccal, rectal, vaginal, inhalation, or topical administration.

30. A method of inhibiting cell division of a pancreatic or an ovarian cancer cell, comprising contacting the cell with a recombinant vector encoding a p84N5 death domain operably linked to a promoter that functions in the cell.

31. A method of inhibiting the growth of a pancreatic or an ovarian cancer cell, comprising contacting the cell with a recombinant vector encoding a p84N5 death domain operably linked to a promoter that functions in the cell.

32. The method of claim 31, wherein the growth is metastatic growth.

33. A method of inhibiting metastatic potential of a pancreatic or an ovarian cancer cell, comprising contacting the cell with a recombinant vector encoding a p84N5 death domain operably linked to a promoter that functions in the cell.

34. A method of reducing tumor burden of a pancreatic or an ovarian cancer cell, comprising contacting the cell with a recombinant vector encoding a p84N5 death domain operably linked to a promoter that functions in the cell.

35. A method of inducing tumor regression of a pancreatic or an ovarian cancer cell, comprising contacting the cell with a recombinant vector encoding a p84N5 death domain operably linked to a promoter that functions in the cell.

36. A method of killing a pancreatic or an ovarian cancer cell, comprising contacting the cell with a recombinant vector encoding a p84N5 death domain operably linked to a promoter that functions in the cell.

37. A method of increasing sensitivity to chemotherapy or radiotherapy of a pancreatic or an ovarian cancer cell, comprising contacting the cell with a recombinant vector encoding a p84N5 death domain operably linked to a promoter that functions in the cell.

38. A method of inducing apoptosis in a pancreatic or an ovarian cancer cell comprising, contacting a cell with a p84N5 protein, peptide, or polypeptide.

39. A method for a cancer therapy comprising,
a) obtaining a biological sample from a cancer patient;
b) detecting the level of expression of a p84N5 polypeptide or protein or peptide in the biological sample;
c) determining the dosage of a radiotherapeutic agent and/or a chemotherapeutic agent to be administered to said patient based on the level of expression of said p84N5 polypeptide or protein or peptide; and
d) providing to said patient the determined dosage of radiotherapeutic agent and/or chemotherapeutic agent.

40. The method of claim 39, further comprising comparing the level of expression of the p84N5 polypeptide or protein or peptide in the biological sample with the level of expression of the p84N5 polypeptide or protein or peptide in a control biological sample.

41. The method according to claim 40, wherein the control biological sample is from a cancer with a known sensitivity to the radiotherapeutic agent or the chemotherapeutic agent.

42. The method of claim 39, wherein the cancer patient is afflicted with a hematological cancer, a thyroid cancer, a melanoma, a T-cell cancer, a B-cell cancer, a breast cancer, an ovarian cancer, a pancreatic cancer, a prostate cancer, a colon cancer, a bladder cancer, a lung cancer, a liver cancer, a stomach cancer, a testicular cancer, an uterine cancer, a brain cancer, a lymphatic cancer, a skin cancer, a bone cancer, a kidney cancer, a rectal cancer, or a sarcoma.

43. A method for a cancer therapy comprising,
a) obtaining a biological sample from a cancer patient;
b) detecting the level of expression of a p84N5 encoding nucleic acid in the biological sample;
c) determining the dosage of a radiotherapeutic agent and/or a chemotherapeutic agent to be administered to said patient based on the level of expression of said p84N5 nucleic acid; and
d) providing to said patient the determined dosage of radiotherapeutic agent and/or chemotherapeutic agent.

44. The method of claim 43, further comprising comparing the level of expression of the p84N5 nucleic acid in the biological sample with the level of expression of the p84N5 nucleic acid in a control biological sample.

45. The method according to claim 44, wherein the control biological sample is from a cancer with a known sensitivity to the radiotherapeutic agent or the chemotherapeutic agent.

46. The method of claim 43, wherein the cancer patient is afflicted with a hematological cancer, a thyroid cancer, a melanoma, a T-cell cancer, a B-cell cancer, a breast cancer, an ovarian cancer, a pancreatic cancer, a prostate cancer, a colon cancer, a bladder cancer, a lung cancer, a liver cancer, a stomach cancer, a testicular cancer, an uterine cancer, a brain cancer, a lymphatic cancer, a skin cancer, a bone cancer, a kidney cancer, a rectal cancer, or a sarcoma.

47. A kit for detecting the levels of a p84N5 polypeptide/peptide/protein in a biological sample comprising;
a) at least one antibody with immunospecificity to a p84N5 polypeptide/peptide/protein;
b) an immunodetection reagent; and
c) reagents and buffers;
enclosed in a suitable container means.

48. A kit for detecting the levels of a p84N5 mRNA in a biological sample comprising;
a) at least two primers that hybridize to regions of SEQ ID NO: 1; and
b) reagents, buffers and enzymes; enclosed in a suitable container means.


SEQUENCE LISTING (13 sequences)

SEQ ID NO: 1 - DNA, 2092 bp, Human
ggccttcgtcgaagatgtctccgacgccgccgctcttcagtttgcccgaagcgcggacgcggtttacgaagtctaccagagaggccttgaacaacaaaaacatcaagccattgttaagtaccttcagccaggtacctggcagtgaaaatgaaaaaaaatgtacccttgaccaagctttcagaggtattctagaagaagaaattataaatcattcatcatgtgaaaacgttttagctattatttctcttgctattgggggagtaactgaaggtatttgtaccgcatctacaccttttgtattgttgggagatgttttggattgtcttcctttggatcagtgtgacacaatattcacttttgtcgaaaaaaatgttgctacttggaaatcaaataccttctatgctgctgggaaaaattacttactacgtatgtgcaatgatctcctaagaagattgtctaaatcccagaatacagtcttctgtggacggattcagctctttttggccaggcttttccctctgtctgagaaatcaggtcttaacttgcagagtcagtttaatctggaaaatgtcactgttttcaatacaaatgagcaggaaagcaccctgggtcagaagcacactgaagatagagaagaaggaatggatgtagaagaaggcgaaatgggagatgaggaagctccaacaacgtgctctattccaattgattacaacctgtatcgaaaattctggtcacttcaggattacttcaggaaccctgtgcaatgctatgagaagatttcatggaaaacttttctcaagtattctgaagaagttttagctgtttttaagagttataaattagatgatactcaggcctcaagaaaaaagatggaagaattgaaaacaggaggagaacatgtatattttgcaaaatttttaacaagtgaaaagctgatggatttacaactgagtgacagtaactttcgtcgacacatcctgttgcagtatctcattttattccaatatctcaaggggcaggtcaaattcaaaagttcaaactatgttttaactgatgagcaatcactttggattgaagatactacaaaatcagtttatcaactactatctgaaaacccccccgatggagaaagattttcaaagatggtagagcatatattaaacactgaagaaaactggaactcgtggaaaaatgaaggttgcccaagttttgtgaaagaaagaacatcagataccaaacctacgagaataattcggaagagaacagcacccgaggacttcctagggaaaggacccaccaaaaaaattctgacgggaaatgaggagttaacaaggctttggaatctttgccctgataatatggaagcctgtaaatcagagacaagggaacacatgcccactttggaggaattctttgaagaagccattgaacaggcagaccctgaaaatatggcggaaaatgaatataaggctatgaacaattcaaattatggttggagagccctgaaactattagcacggagaagccctcacttcttccagccaaccaaccagcagtttaaaagtttacaagaatatcttgaaaatatggtaataaagctagccaaggaattaccgcctccttctgaagaaataaaaacaggtgaggatgaagatgaggaagataatgatgctctactgaaggaaaatgaaagtcctgatgttcggcgagacaaacctgtaacaggagaacaaatagaggtatttgccaacaagctgggtgaacaatggaagattctggctccctacttggaaatgaaagactcagaaattaggcagattgagtgtgacagtgaagacatgaagatgagagctaagcagctcctggttgcctggcaagatcaagagggagttcatgcaacacctgagaatctgattaatgcactgaataagtctggattaagtgaccttgcagaaagtctaactaatgacaatgagacaaatagttagcttcttttttttttctttttattaaaactgtgatagattttgttaccaagcagcatttgataagaggtccactggttttggtaaacaataaacatttttataac

SEQ ID NO: 2 - protein, 657 aa, Human
MSPTPPLFSLPEARTRFTKSTREALNNKNIKPLLSTFSQVPGSENEKKCTLDQAFRGILEEEIINHSSCENVLAIISLAIGGVTEGICTASTPFVLLGDVLDCLPLDQCDTIFTFVEKNVATWKSNTFYAAGKNYLLRMCNDLLRRLSKSQNTVFCGRIQLFLARLFPLSEKSGLNLQSQFNLENVTVFNTNEQESTLGQKHTEDREEGMDVEEGEMGDEEAPTTCSIPIDYNLYRKFWSLQDYFRNPVQCYEKISWKTFLKYSEEVLAVFKSYKLDDTQASRKKMEELKTGGEHVYFAKFLTSEKLMDLQLSDSNFRRHILLQYLILFQYLKGQVKFKSSNYVLTDEQSLWIEDTTKSVYQLLSENPPDGERFSKMVEHILNTEENWNSWKNEGCPSFVKERTSDTKPTRIIRKRTAPEDFLGKGPTKKILTGNEELTRLWNLCPDNMEACKSETREHMPTLEEFFEEAIEQADPENMAENEYKAMNNSNYGWRALKLLARRSPHFFQPTNQQFKSLQEYLENMVIKLAKELPPPSEEIKTGEDEDEEDNDALLKENESPDVRRDKPVTGEQIEVFANKLGEQWKILAPYLEMKDSEIRQIECDSEDMKMRAKQLLVAWQDQEGVHATPENLINALNKSGLSDLAESLTNDNETNS

SEQ ID NO: 3 - DNA, 34 bp, Artificial Sequence
  Description of Artificial Sequence  Synthetic Primer
cttgatcttgcsrggcaaccrsgagctgcttagc

SEQ ID NO: 4 - DNA, 21 bp, Artificial Sequence
  Description of Artificial Sequence  Synthetic Primer
agggagttcatgcaacacctg

SEQ ID NO: 5 - DNA, 21 bp, Artificial Sequence
  Description of Artificial Sequence  Synthetic Primer
tcatgtcttcactgtcacact

SEQ ID NO: 6 - DNA, 29 bp, Artificial Sequence
  Description of Artificial Sequence  Synthetic Primer
aattattctcgtaggtttggtatctgatg

SEQ ID NO: 7 - DNA, 30 bp, Artificial Sequence
  Description of Artificial Sequence  Synthetic Primer
attctgacgggaaatgaggagttaacaagg

SEQ ID NO: 8 - DNA, 42 bp, Artificial Sequence
  Description of Artificial Sequence  Synthetic Primer
catctcctgggcataacgaattattctcgtaggtttggtatc

SEQ ID NO: 9 - DNA, 42 bp, Artificial Sequence
  Description of Artificial Sequence  Synthetic Primer
gaaggcgaagaagaagccattctgacgggaaatgaggagtta

SEQ ID NO: 10 - DNA, 39 bp, Artificial Sequence
  Description of Artificial Sequence  Synthetic Primer
gaacttcttcgtcataattattctcgtaggtttggtatc

SEQ ID NO: 11 - DNA, 36 bp, Artificial Sequence
  Description of Artificial Sequence  Synthetic Primer
ggcacgctcacgatcattctgacgggaaatgaggag

SEQ ID NO: 12 - protein, 17 aa, Artificial Sequence
  Description of Artificial Sequence  Synthetic Peptide
RKRTAPEDFLGKGPTKK

SEQ ID NO: 13 - protein, 12 aa, Artificial Sequence
  Description of Artificial Sequence  Synthetic Peptide
RYAQEMEGEEEA